(19) United States
(12) Patent Application Publication
(10) Pub. No.: US 2018/0127465 A1
(43) Pub. Date: May 10, 2018

(54) MULTIFUNCTIONAL OPIOID RECEPTOR LIGANDS AND METHODS OF TREATING PAIN

(71) Applicant: THE ARIZONA BOARD OF REGENTS ON BEHALF OF THE UNIVERSITY OF ARIZONA, Tucson, AZ (US)

(72) Inventors: Yeon Sun Lee, Tucson, AZ (US); Victor J. Hruby, Tucson, AZ (US); Frank Porreca, Tucson, AZ (US)

(21) Appl. No.: 15/820,133

(22) Filed: Nov. 21, 2017

Related U.S. Application Data
(63) Continuation-in-part of application No. PCT/US16/33529, filed on May 20, 2016.
(60) Provisional application No. 62/165,063, filed on May 21, 2015.
(60) Provisional application No. 62/476,980, filed on Mar. 27, 2017.

(51) Int. Cl. C07K 5/097; C07K 5/065

(52) U.S. Cl. CPC C07K 5/0823; C07K 5/06078

(57) ABSTRACT

Opioid receptor ligands (ORLs) that are multifunctional having agonist activity at mu opioid receptor (MOR), agonist activity at delta opioid receptor (DOR), and antagonist (or partial agonist) activity at kappa opioid receptor (KOR). The ORLs comprise peptide portions that are analogs derived from enkephalins, EM-1, or DALDA, as well as tail portions that comprise a lipophilic molecule such as a 4-anilidopiperidine moiety.

### CROSS REFERENCE

This application claims priority to U.S. Patent Application No. 62/165,063 filed May 21, 2015, PCT/US16/33529 filed May 20, 2016, and U.S. Patent Application 62/476,980 filed Mar. 27, 2017, the specification(s) of which is/are incorporated herein in their entirety by reference.

### GOVERNMENT SUPPORT

This invention was made with government support under Grant No. P01 DA006284, awarded by NIH. The government has certain rights in the invention.

### FIELD OF THE INVENTION

The present invention relates to ligands for mu, delta, and kappa opioid receptors, more particularly to multifunctional opioid peptides that function as mu opioid receptor (MOR), delta opioid receptor (DOR) agonists, and kappa opioid receptor (KOR) antagonists (or partial agonists). The present invention also relates to treating pain or other conditions using the multifunctional opioid peptides herein. The present invention also relates to pharmacophores for modifying C-terminal regions of opioid peptides (e.g., enkephalins, DALDA, EM-1, EM-2, etc.) for conferring particular KOR activity.

### BACKGROUND OF THE INVENTION

Opioids are commonly used in the treatment of severe pain. Opioids have analgesic activity through their interaction with the opioid receptors (e.g., mu (μ) opioid receptor (MOR), delta (δ) opioid receptor (DOR), kappa (κ) opioid receptor (KOR)), mostly with MOR. However, the clinical use of opioids is limited by associated side effects such as respiratory depression, constipation, development of tolerance, and addiction. Indeed, chronic pain and subsequence chronic administration of a MOR agonist can lead to KOR activation, which results in undesirable adverse and addictive behaviors. For this reason, a KOR antagonist (or partial agonist) could be used to reduce such undesirable effects of chronic MOR activation.

Inventors have surprisingly discovered opioid peptides, e.g., opioid receptor ligands (ORLs) that are multifunctional, e.g., acting as MOR agonists, DOR agonists, and KOR antagonists (or partial agonists). Without wishing to limit the present invention to any theory or mechanism, it is believed that this MOR/DOR agonist with KOR antagonist/partial agonist activity encompassed by a single molecule may be better and/or more effective than using co-administration of two or more molecules to achieve MOR/DOR agonist and KOR antagonist/partial agonist activity.

In some embodiments, the multifunctional ORLs may comprise peptide analogs derived from enkephalins. Enkephalins are pentapeptides (peptides containing 5 amino acids) that are endogenous ligands of the opioid receptors (e.g., MOR, and DOR). There are two known forms of enkephalins: leucine-containing enkephalin (Leu-Enk, or YGGFL (SEQ ID NO: 1)) and methionine-containing enkephalin (Met-Enk, or YGGFM (SEQ ID NO: 2)). In some embodiments, the multifunctional ORLs comprise peptide analogs derived from endomorphin-1 (EM-1), endomorphin-2 (EM-2) or other opioid ligands such as DALDA, FE20066, etc.

In some embodiments, the ORLs comprise a 4-anilidopiperidine moiety, e.g., fentanyl analog, an analog of a 4-anilidopiperidine, etc., e.g., N-phenyl-N-piperidin-4-ylpropionamide (Ppp).

The present invention also provides C-terminal modifications (pharmacophores) that confer KOR antagonist activity to opioid peptides. The present invention also provides modifications, such as halogenation of a phenylalanine of a Ppp moiety, that confer KOR antagonist or partial agonist activity. Those kappa activities may help reduce KOR- or MOR-related side effects. For example modifications of opioid ligands (such as DALDA, EM-1, EM-2, and FE20066) with pharmacophores (e.g., the C-terminal modifications of the aforementioned molecules) may generate the same KOR activity.

For example, the present invention provides ORLs with a Ppp tail, wherein the Ppp comprises an R group (e.g., a halogen).

### SUMMARY OF THE INVENTION

The present invention features multifunctional opioid receptor ligands (ORLs) and methods of use of said multifunctional ORLs.

In certain embodiments, the ORLs herein have agonist activity at mu opioid receptor (MOR), agonist activity at delta opioid receptor (DOR), and antagonist activity at kappa opioid receptor (KOR). In certain embodiments, the ORLs herein have agonist activity at mu opioid receptor (MOR), agonist activity at delta opioid receptor (DOR), and partial agonist activity at kappa opioid receptor (KOR).

The present invention provides multifunctional opioid receptor ligands (ORLs) according to Formula 1: Aaa-Bbb-Ccc-Ddd(X)-Eee.

In some embodiments, Aaa is selected from 2′-6′-dimethyltyrosine (Dmt), Tyrosine (Tyr), Tmt, Phe, Dmp, and Mdp; Bbb is selected from D-Alanine (D-Ala), Alanine (Ala), D-Norleucine (D-Nle), Norleucine (Nle), Proline (Pro), Arginine (Arg), D-Arginine (D-Arg), tetrahydroisoquinoline-3-carboxylic acid (Tic), and D-Tic; Ccc is selected from Gly, Phenylalanine(X) (Phe(X)), Trp, and naphthylalanine (Nal) or is absent; Ddd(X) is Gly, Phe(X), Trp, Nal, or Lys; and Eee comprises N-phenyl-N-piperidin-4-ylpropionamide-R (Ppp(R)); wherein X and R both comprise a halogen. In some embodiments, X is selected from H, F, Cl, and Br. In some embodiments, X is selected from H, F, and Cl. In certain embodiments, R is selected from 3-Cl, 4-Cl, 3-F, 4-F, and 2,4-diCl.

In some embodiments, Aaa is selected from 2′-6′-dimethyltyrosine (Dmt), Tyrosine (Tyr), Tmt, Phe, Dmp, and Mdp; Bbb is selected from D-Alanine (D-Ala), D-Norleucine (D-Nle), and D-Tic; Ccc is selected from Gly, Phenylalanine(X) (Phe(X)), Trp, and naphthylalanine (Nal) or is absent; Ddd(X) is Gly, Phe(X), Trp, Nal, or Lys; and Eee comprises N-phenyl-N-piperidin-4-ylpropionamide-R (Ppp(R)); wherein X and R both comprise a halogen. In some embodiments, X is selected from H, F, Cl, and Br. In some embodiments, X is selected from H, F, and Cl. In certain embodiments, R is selected from 3-Cl, 4-Cl, 3-F, 4-F, and 2,4-diCl.

In some embodiments, Aaa is selected from Tmt, Phe, Dmp, and Mdp; Bbb is selected from D-Alanine (D-Ala), Alanine (Ala), D-Norleucine (D-Nle), Norleucine (Nle), Proline (Pro), Arginine (Arg), D-Arginine (D-Arg), tetrahydroisoquinoline-3-carboxylic acid (Tic), and D-Tic; Ccc is selected from Gly, Phenylalanine(X) (Phe(X)), Trp, and naphthylalanine (Nal) or is absent; Ddd(X) is Gly, Phe(X), Trp, Nal, or Lys; and Eee comprises N-phenyl-N-piperidin-4-ylpropionamide-R (Ppp(R)); wherein X and R both comprise a halogen. In some embodiments, X is selected from H, F, Cl, and Br. In some embodiments, X is selected from H, F, and Cl. In certain embodiments, R is selected from 3-Cl, 4-Cl, 3-F, 4-F, and 2,4-diCl.

In some embodiments, Aaa is selected from 2′-6′-dimethyltyrosine (Dmt), Tyrosine (Tyr), Tmt, Phe, Dmp, and Mdp; Bbb is selected from Alanine (Ala), Norleucine (Nle), Proline (Pro), Arginine (Arg), tetrahydroisoquinoline-3-carboxylic acid (Tic); Ccc is selected from Gly, Phenylalanine(X) (Phe(X)), Trp, and naphthylalanine (Nal) or is absent, wherein X is a halogen; Ddd(X) is Gly, Phe(X), Trp, Nal, or Lys, wherein X is a halogen; and Eee is a 4-anilidopiperidine moiety (e.g., Ppp). In some embodiments, Ppp comprises Ppp(R), wherein R comprises a halogen. In some embodiments, R is selected from 3-Cl, 4-Cl, 3-F, 4-F, and 2,4-diCl. In some embodiments, X is selected from H, F, Cl, and Br.

The present invention also provides multifunctional opioid receptor ligands (ORLs) according to a Formula 3: Aaa-Bbb-Ccc-Phe(Br)-Eee.

In certain embodiments, Aaa is selected from 2′-6′-dimethyltyrosine (Dmt), Tyrosine (Tyr), Tmt, Phe, Dmp, and Mdp; Bbb is selected from D-Alanine (D-Ala), Alanine (Ala), D-Norleucine (D-Nle), Norleucine (Nle), Proline (Pro), Arginine (Arg), D-Arginine (D-Arg), tetrahydroisoquinoline-3-carboxylic acid (Tic), and D-Tic; Ccc is selected from Gly, Phenylalanine(X) (Phe(X)), Trp, and naphthylalanine (Nal) or is absent, wherein X is a halogen; and Eee is a 4-anilidopiperidine moiety (e.g., Ppp). In some embodiments, Ppp comprises Ppp(R), wherein R comprises a halogen. In some embodiments, R is selected from 3-Cl, 4-Cl, 3-F, 4-F, and 2,4-diCl. In some embodiments, X is selected from H, F, Cl, and Br.

In certain embodiments, Aaa is selected from 2′-6′-dimethyltyrosine (Dmt), Tyrosine (Tyr); Bbb is selected from D-Alanine (D-Ala), Alanine (Ala), D-Norleucine (D-Nle), Norleucine (Nle), Proline (Pro), Arginine (Arg), D-Arginine (D-Arg), tetrahydroisoquinoline-3-carboxylic acid (Tic), and D-Tic; Ccc is selected from Gly, Phenylalanine(X) (Phe(X)), Trp, and naphthylalanine (Nal) or is absent, wherein X is a halogen; and Eee is a 4-anilidopiperidine moiety (e.g., Ppp). In some embodiments, Ppp comprises Ppp(R), wherein R comprises a halogen. In some embodiments, R is selected from 3-Cl, 4-Cl, 3-F, 4-F, and 2,4-diCl. In some embodiments, X is selected from H, F, Cl, and Br.

The present invention also provides multifunctional opioid receptor ligands (ORLs) according to Formula 4: Aaa-Bbb-Ccc-Ddd(X)Yyy(n)-Eee.

In some embodiments, Aaa is selected from 2′-6′-dimethyltyrosine (Dmt), Tyrosine (Tyr), Tmt, Phe, Dmp, and Mdp; Bbb is selected from D-Alanine (D-Ala), D-Norleucine (D-Nle), Proline (Pro), and D-Arginine (D-Arg), tetrahydroisoquinoline-3-carboxylic acid (Tic), D-Tic; Ccc is selected from Gly, Phenylalanine(X) (Phe(X)), Trp, and naphthylalanine (Nal) or is absent, wherein X is a halogen; Ddd(X) is Gly, Phe(X), Trp, Nal, or Lys, wherein X is a halogen; Yyy is selected from one or a combination of Leu, Arg, Met, Lys, or lie; and Eee is a 4-anilidopiperidine moiety (e.g., Ppp). In some embodiments, Ppp comprises Ppp(R), wherein R comprises a halogen. In some embodiments, R is selected from 3-Cl, 4-Cl, 3-F, 4-F, and 2,4-diCl. In some embodiments, X is selected from H, F, Cl, and Br. In some embodiments, n=1. In some embodiments, n=2. In some embodiments, n=3. In some embodiments, n=4. In some embodiments, n=5. In some embodiments, n=6. In some embodiments, n=7. In some embodiments, n=8. In some embodiments, n is 8 or more, e.g., 9, 10, 11, 12, 13, etc.

The present invention also provides multifunctional opioid receptor ligands (ORLs) according to Formula 5: Aaa-DArg-Ccc-Ddd-Eee.

In some embodiments, Aaa is selected from Tyr or 2′-6′-dimethyltyrosine (Dmt); Ccc is selected from Phe, Phe(X), or 1-naphthylalanine (1Nal); Ddd is selected from Lys, Gly or is absent; Eee is a 4-anilidopiperidine moiety; and X is selected from F, Cl, or Br. In some embodiments, the 4-anilidopiperidine moiety comprises Ppp. In some embodiments, Ppp comprises Ppp(R), wherein R comprises a halogen. In some embodiments, R is selected from 3-Cl, 4-Cl, 3-F, 4-F, and 2,4-diCl. In some embodiments, the ORL is SEQ ID NO: 21, SEQ ID NO: 22, SEQ ID NO: 23, SEQ ID NO; 24, SEQ ID NO: 25, SEQ ID NO: 26, SEQ ID NO: 42, or SEQ ID NO: 43.

The present invention also provides multifunctional opioid receptor ligands (ORLs) according to Formula 6: Aaa-Pro-Ccc-Phe(X)-Eee.

In some embodiments, Aaa is selected from Tyr or 2′-6′-dimethyltyrosine (Dmt); Ccc is selected from Trp, Phe, Gly, or Phe(X); Eee is a 4-anilidopiperidine moiety (e.g., Ppp), and X is selected from F, Cl, or Br. In some embodiments, Ppp comprises Ppp(R), wherein R comprises a halogen. In some embodiments, R is selected from 3-Cl, 4-Cl, 3-F, 4-F, and 2,4-diCl. In some embodiments, the ORL is selected from SEQ ID NO: 27, SEQ ID NO: 28, SEQ ID NO: 29, or SEQ ID NO: 30.

The present invention also provides multifunctional opioid receptor ligands (ORLs) according to Formula 7: DPhe-DPhe-DNle-Ddd-Eee.

In some embodiments, Ddd is selected from D-Arg or D-Lys, and Eee is a 4-anilidopiperidine moiety (e.g., Ppp). For example, in some embodiments, Ddd is D-Arg and Eee is Ppp. In some embodiments, Ddd is D-Lys and Eee is Ppp. In some embodiments, Ppp comprises Ppp(R), wherein R comprises a halogen. For example, in some embodiments, Ddd is D-Arg and Eee is Ppp(R), wherein R comprises a halogen (e.g., Cl, F, Br). In some embodiments, Ddd is D-Lys and Eee is Ppp(R), wherein R comprises a halogen (e.g., Cl, F, Br). In some embodiments, R is selected from 3-Cl, 4-Cl, 3-F, 4-F, and 2,4-diCl. In some embodiments, the ORL is according to SEQ ID NO: 44.

The present invention also provides an opioid receptor ligand dimer according to SEQ ID NO: 19. The present invention also provides an opioid receptor ligand dimer according to SEQ ID NO: 20.

The present invention also provides methods of reducing pain, e.g., reducing pain in a subject in need of a KOR antagonist or KOR partial agonist. In some embodiments, the method comprises identifying a subject in need of a kappa opioid receptor (KOR) antagonist or partial agonist; and introducing to the subject a multifunctional ORL according the present invention, wherein the ORL is effective for reducing pain.

The present invention also features methods of blocking kappa opioid receptor. In some embodiments, the method comprises introducing to the KOR a multifunctional ORL according to the present invention.

The present invention also features methods of blocking KOR, activating MOR, and activating DOR in a subject. In some embodiments, the method comprises introducing to the subject a multifunctional ORL according to the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

This patent application contains at least one drawing executed in color. Copies of this patent or patent application publication with color drawing(s) will be provided by the Office upon request and payment of the necessary fee.

FIG. 1 shows the well-known structure-activity relationship (SAR) results of Dynorphin A (Dyn A (SEQ ID NO: 3)) and Enkephalins for opioid activities. Peptides tested include Dyn A (SEQ ID NO: 3), an endogenous KOR ligand, a peptide containing the first 13 amino acids of Dyn A (Dyn A 1-13 (SEQ ID NO: 4)), a peptide containing the first 8 amino acids of Dyn A (Dyn A 1-8 (SEQ ID NO: 5)), Dyn B (SEQ ID NO: 6), Leu-Enk (SEQ ID NO: 1), and Met-Enk (SEQ ID NO: 2).

FIG. 2 shows non-limiting examples of anilidopiperidine analogs as tails of the ORLs of the present invention.

FIG. 3 shows GTPγS activity of LYS739 (SEQ ID NO: 10), U50,488, and Naloxone at KOR. U50,488 is known to have agonist activity at KOR. Naloxone is known to have antagonist activity at KOR. LYS739 (SEQ ID NO: 10) appears to have partial agonist/antagonist activities at KOR.

FIG. 4A shows [3S]GTPγS assays: MOR (left) and DOR (right) antagonist modes. LYS739 (SEQ ID NO: 10), LYS744 (SEQ ID NO: 15), and MR115 (SEQ ID NO: 28) do not possess antagonist activity at MOR and DOR.

FIG. 4B shows [35S]GTPγS assays: KOR agonist (left) and antagonist (right) modes. LYS540 (SEQ ID NO: 9), LYS644 (SEQ ID NO: 14), and MR121 (SEQ ID NO: 126) are partial agonist/antagonist at KOR. CYF132 (SEQ ID NO: 13) is observed as a partial agonist at KOR.

FIG. 5A and FIG. 5B show the effects of fentanyl analogs, LYS436, LYS739 and LYS416 and biphalin on H/A and reoxygenation challenge. For both the graphs; ‘*’ compared to no drug treated group; ‘#’ compared to biphalin treated group; *p<0.05, ***p<0.001, ***p<0.0001; #p<0.05, ##p<0.01; data from 3 to 4 independent primary neuron isolations with 2-3 replicates treatment per isolation. Compared to normoxic and 0.1% tritonX, all experimental groups were significantly different (p<0.0001). A) MTT assay: Effect of fentanyl analogs LYS436, LYS739 and LYS416 and biphalin on 3 hr H/A ad 24 hr reoxygenation. Compared to no drug treated group, LYS436 (p<0.0001), LYS739 (p<0.0001), LYS416 (p<0.0001), biphalin (p<0.001) and fentanyl (p<0.05) significantly increased neuronal survival. Again, compared to biphalin, LYS739 (p<0.01) and LYS416 (p<0.05) showed better neuroprotection in terms of neuronal cell survival. LYS436 (p<0.05), LYS739 (p<0.0001) and LYS416 (p<0.001) demonstrated better neuronal survival compared to fentanyl alone. NTX reversed the effect of LYS436, LYS739, LYS416 and biphalin. B) LDH assay: Relative neuronal death in terms of LDH production was assessed upon 3 hr H/A and 24 hr reoxygenation. Fentanyl analogs LYS436 (p<0.001), LYS739 (p<0.0001) and LYS416 (p<0.0001) and biphalin (p<0.001) and fentanyl (p<0.05) significantly decreased neuronal cell death compared to no drug treated group. LYS739 (p<0.05) significantly decreased neuronal cell death in comparison to biphalin. LYS739 (p<0.001) and LYS416 (p<0.01) showed better neuroprotection compared to fentanyl alone. NTX reversed the effect of LYS436, LYS739, LYS416 and biphalin.

FIG. 6A and FIG. 6B show the effects of fentanyl analogs, LYS436, LYS739 and LYS416 and biphalin on NMDA challenge. For both the graphs; ‘*’ compared to no drug treated group; ‘#’ compared to biphalin treated group; *p<0.05, *p<0.01 ***p<0.001, ****p<0.0001; #p<0.05, ##p<0.01; data from 3 to 4 independent primary neuron isolations with 2-3 replicates treatment per isolation. All experimental groups were significantly different (p<0.0001) compared to normoxia and 0.1% tritonX. A) MTT assay: effects of fentanyl analogs and biphalin (10 nM) on primary cortical neuron with NMDA (50 uM) exposure for 3 hr assessed by relative neuronal survival. LYS436 (p<0.0001), LYS739 (p<0.0001), LYS416 (p<0.001), biphalin (p<0.01) and fentanyl (p<0.05) significantly improved relative neuronal survival compared to no drug treated group. Effect of LYS739 (p<0.01) and LYS436 (p<0.05) were significantly better than biphalin. LYS436 (p<0.01) and LYS739 (p<0.001) also increased neuronal survival when compared to fentanyl alone. NTX reversed the effect of LYS436, LYS739, LYS416 and biphalin. B) LDH assay: effects of fentanyl analogs and biphalin (10 nM) on primary cortical neuron with NMDA (50 uM) exposure for 3 hr assessed by relative neuronal death. In comparison to no drug treated group, LYS436 (p<0.0001), LYS739 (p<0.0001), LYS416 (p<0.01), biphalin (p<0.0001) and fentanyl (p<0.05) significantly decreased relative neuronal death. LYS739 (p<0.05) and LYS436 (p<0.05) showed better neuroprotection compared to biphalin. Compared to fentanyl alone, LYS436 (p<0.0001) and LYS739 (p<0.0001) displayed better neuroprotection in terms of LDH production. NTX reversed the effect of LYS436, LYS739, LYS416, biphalin and fentanyl.

FIG. 7 shows the effects of fentanyl analogs and biphalin on primary cortical neuronal ROS production upon exposure to 3 hr H/A and 24 hr reoxygenation. (‘*’ compared to no drug treated group; ‘#’ compared to biphalin treated group; *p<0.05, **p<0.01 ***p<0.001, #p<0.05; data from 3 to 4 independent primary neuron isolations with 2-3 replicates treatment per isolation). All experimental groups were significantly different compared to normoxia (p<0.0001) and H2O2 (p<0.001). LYS436 (p<0.001), LYS739 (p<0.001), LYS416 (p<0.01) and biphalin (p<0.05) significantly decreased ROS production compared to no drug treated group. LYS739 (p<0.05) showed better neuroprotection compared to biphalin in terms of ROS production. In comparison to fentanyl alone, LYS436 (p<0.001) and LYS739 (p<0.001) significantly reduced ROS production. NTX reversed the effect of biphalin, LYS436, LYS739 and LYS416.

FIGS. 8A, 8B, and 8C show the effects of fentanyl analog LYS739 and biphalin (5 mg/kg, I.P. administration, 10 min after reperfusion), fentanyl (0.2 mg/kg, I.P. administration, 10 min after reperfusion) and non-selective OR antagonist NTX (1 mg/kg, I.P. administration, 10 min before surgery) or vehicle (0.9% saline) on edema and infarct formation in transient MCAO (60 min occlusion and 24 hr reperfusion). A) Representative TTC staining of brain slices from vehicle and drug treated mice. B) Brain edema ratio of brain in vehicle and drug treated groups. Fentanyl analog LYS739 (p<0.05) and biphalin (p<0.05) significantly decreased edema formation compared to vehicle treated group. In comparison to fentanyl alone, both LYS739 (p<0.05) and biphalin (p<0.05) significantly reduced edema formation. NTX reversed the effect of both biphalin (p<0.05) and LYS739 (p<0.05). NTX and FENT alone did not show any significant effect compared to vehicle treated group. C) Brain infarct ration in vehicle and drug treated mice. In comparison to vehicle treated group, fentanyl analog LYS739 (p<0.0001) and biphalin (p<0.0001) significantly reduced infarct formation in mice. Fentanyl and NTX alone did not show any improvement compared to saline treated group. Both biphalin (p<0.0001) and LYS739 (p<0.0001)) decreased infarct formation compared to fentanyl alone. NTX reversed the effect of biphalin (p<0.0001) and LYS739 (p<0.0001). (‘*’ compared to vehicle treated group; *p<0.05; ****p<0.0001; numbers indicated in the parenthesis in the figure columns denote to the number of experimental animals per group).

FIG. 9 shows the neurological score evaluation of mice 24 hr after ischemia and drug treatment. Both biphalin (p<0.05) and LSY739 (p<0.05) improved neurological behavior compared to vehicle treated group whereas FENT and NTX alone did not improve any neurological score compared to vehicle treated group. NTX reversed the effect of biphalin (p<0.05) and fentanyl analog LYS739 (p<0.05). ‘*’ compared to vehicle treated group; *p<0.05; numbers indicated in the parenthesis in the figure columns denote to the number of experimental animals per group).

FIG. 10A-10E shows in vivo assays wherein bilateral RVM or intrathecal (i.th.) injections of LYS739 (10 ug/0.5 uL) reversed tactile allodynia and thermal hyperalgesia in the Hargreaves test and the von Frey test, respectively.

FIG. 11 shows stability of LYS739 in human plasma, e.g., HPLC profiles after incubation at 37C and peptide concentration (%) at various times.

FIG. 12 shows non-limiting examples of multifunctional enkephalin analogues.

FIG. 13 shows an example of the design of multifunctional opioid ligands with MOR/DOR agonist and KOR antagonist activity.

FIG. 14 shows an example of a scheme for the synthesis of multifunctional opioid analogs. (i) Boc-amino acid/BOP/HOBt/NMM (1.1 eq/1.1 eq/1.1 eq/2 eq) in DMF for 3 h at RT. (ii) 100% TFA for 20 min at 0° C. (iii) RP-HPLC: 10-50% of acetonitrile within 20 min. BOP: (Benzotriazol-1-yloxy)tris(dimethylamino) phosphoniumhexafluorophosphate; HOBt: 1-hydroxybenzotriazole; NMM: N-methyl morpholine. See FIG. 13 for Aaa, Bbb, Ccc, Ddd, and R.

FIG. 15 shows modifications of multifunctional ligands with MOR/DOR agonist and KOR antagonist activity.

FIG. 16 shows ligand modifications for enhanced KOR activity.

FIG. 17 shows an example of a scheme for the synthesis of multifunctional enkephalin analogs. (i) Boc-amino acid/BOP/HOBt/NMM (1.1 eq/1.1 eq/1.1 eq/2 eq) in DMF for 2-4 h at RT. (ii) 100% TFA for 20 min at 0° C. (iii) RP-HPLC: 10-50% of acetonitrile within 20 min. (iv) Boc-cysteineBOP/HOBt/NMM (2.2 eq/2.2 eq/4 eq) in DMF for 3-5 h at RT (v) 5% TFA, 10 min, RT. (vi) DCC/HOBt/DIPEA. DCC: N,N′-dicyclohexylcarbodiimide; BOP: (Benzotriazol-1-yloxy)tris(dimethylamino) phosphoniumhexafluorophosphate; HOBt: 1-hydroxybenzotriazole; NMM: N-methyl morpholine. Mtt: 4-methyltrityl. M: 0, 1. See FIG. 15 for I, R, X, Fff, and Ggg.

### DESCRIPTION OF PREFERRED EMBODIMENTS

The present invention features multifunctional opioid receptor ligands (ORLs), acting as MOR agonists, DOR agonists, and KOR antagonists (or partial agonists). The present invention also features methods of use of said multifunctional ORLs, e.g., methods of treating pain or other conditions using peptides of the present invention.

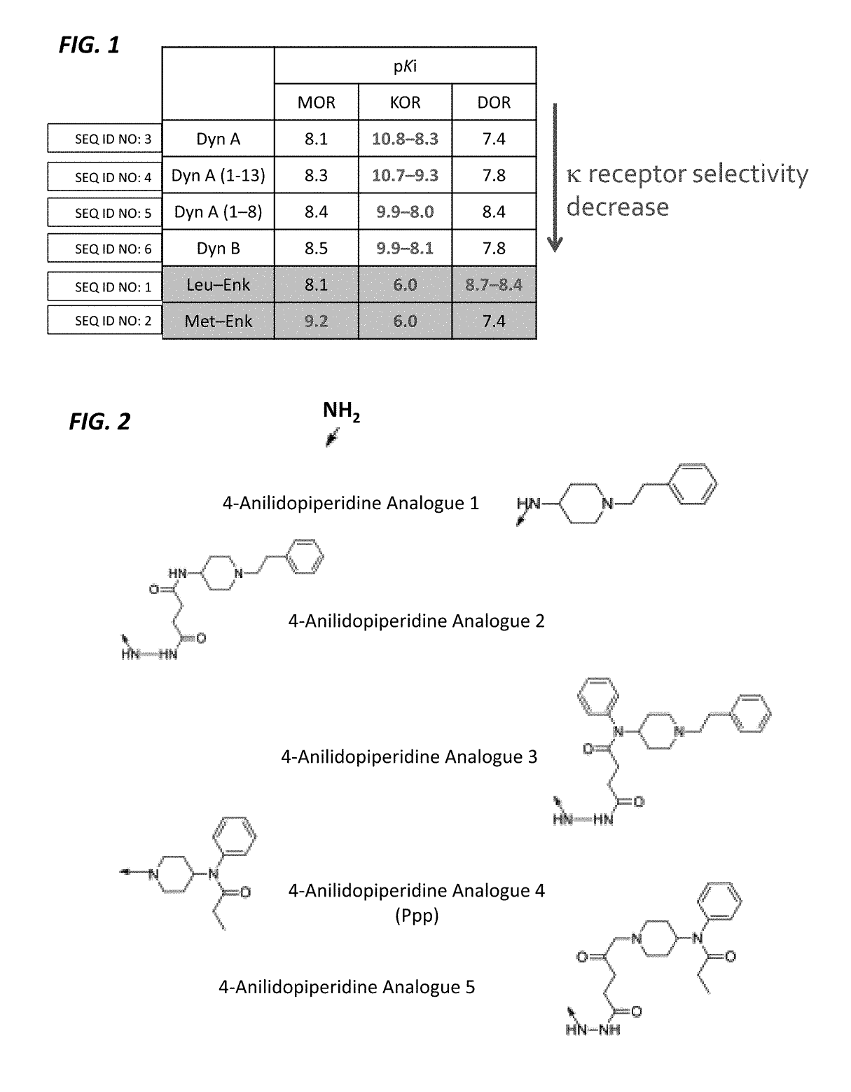

FIG. 1 shows the well-known structure-activity relationship (SAR) results of Dynorphin A (Dyn A) and enkephalins for opioid activities. Enkephalins shown are Leu-Enk (YGGFL, SEQ ID NO: 1) and Met-Enk (YGGFM, SEQ ID NO: 2). Dyn A is an endogenous kappa opioid receptor (KOR) ligand. The sequence for Dyn A is YGGFLRRIRPKLKWDNQ (SEQ ID NO: 3). (Note that the first five amino acids of Dyn A is Leu-Enk). Other peptides tested include a peptide containing the first 13 amino acids of Dyn A (Dyn A 1-13, YGGFLRRIRPKLK (SEQ ID NO: 4)), a peptide containing the first 8 amino acids of Dyn A (Dyn A 1-8, YGGFLRRI (SEQ ID NO: 5)), and Dyn B (YGGFLRRNFLVVT (SEQ ID NO: 6)). Without wishing to limit the present invention to any theory or mechanism, it appears that KOR selectivity decreases as the C-terminal residues of Dyn A are removed (e.g., Dyn A is more selective for KOR than is Leu-Enk). Without wishing to limit the present invention to any theory or mechanism, it is thought that residues following the first four amino acids of enkephalin, e.g., the residue(s) following the Phe/F residue of the enkephalin (or derivative) may be a region that helps make the ORL active for KOR, e.g., the residues following the first four amino acids of the enkephalin (or derivative thereof) may provide specificity for KOR.

The ORLs of the present invention comprise a peptide portion, e.g., a peptide analog derived from enkephalins (e.g., Leu-Enk (YGGFL, SEQ ID NO: 1) or Met-Enk (YGGFM, SEQ ID NO: 2)) and a tail portion linked to the C-terminus of the peptide portion. In some embodiments, the peptide portion comprises four residues (e.g., amino acids, analogs or derivatives thereof), occupying position 1, 2, 3, and 4. In some embodiments, the peptide portion comprises three residues (e.g., amino acids, analogs or derivatives thereof), occupying position 1, 2, and 4. The peptide portion may be based on the enkephalin sequence e.g., Leu-Enk (YGGFL, SEQ ID NO: 1) or Met-Enk (YGGFM, SEQ ID NO: 2).

In some embodiments, the tail portion comprises a lipophilic molecule (e.g., a 4-anilidopiperidine moiety), e.g., the tail portion may comprise a residue or compound that increases the lipophilicity of the peptide portion. In some embodiments, the tail comprises a N-phenyl-N-piperidin-4-ylpropionamide (Ppp) moiety. In some embodiments, the tail comprises —NH₂. Other non-limiting examples of tail portion molecules (tail compounds) are shown in FIG. 2. For reference, Lee et al. (Bioorganic and Medicinal Chemistry Letters 17, 2007, pp 2161-2165) describes 4-anilidopiperidine analogues for biological activities at mu and delta opioid receptors.

Various non-limiting examples of formulas are presented herein for ORLs. For example, the present invention provides ORLs according to Formula 1 (Aaa-DBbb-Ccc-Ddd(X)-Eee). In some embodiments, Aaa is selected from 2′-6′-dimethyltyrosine (Dmt) and Tyrosine (Tyr). In some embodiments, D-Bbb is selected from D-Alanine (D-Ala), D-Norleucine (D-Nle), Proline (Pro), and D-Arginine (D-Arg); In some embodiments, Ccc is selected from Gly, Phenylalanine(X) (Phe(X)), and naphthylalanine (Nal) or is absent. In some embodiments, Ddd(X) is Gly, Phe(X), or Lys. Eee is a tail portion, wherein the tail portion is lipophilic. In some embodiments, X is Br. In some embodiments, X is selected from H, F, Cl, and Br. In some embodiments, Eee is selected from —NH₂ and a 4-anilidopiperidine moiety. In some embodiments, the 4-anilidopiperidine moiety comprises N-phenyl-N-piperidin-4-ylpropionamide (Ppp). The present invention is not limited to Formula 1. Dmt refers to 2′-6′-dimethyltyrosine, DXxx refers to a D amino acid, and X refers to a halogen or other appropriate compound, e.g., H, Cl, F, or a methyl group. N-phenyl-N-piperidin-4-ylpropionamide may be abbreviated as Ppp. In some embodiments, residue 1 (e.g., Dmt, Aaa, etc.) comprises Dmt or Tyr. In some embodiments, residue 2 (DXxx, Bbb, etc.) comprises DAla, DNle (D-norleucine), Pro, or DArg. In some embodiments, residue 3 (Gly, Ccc, etc.) comprises Gly, Phe, Phe(X), or Nal, wherein X may refer to H, Cl, F, methyl group, or any other appropriate modification of Phe. In some embodiments, residue 3 is absent. In some embodiments, residue 4 (Phe(X), Ddd, etc.) comprises Gly, Phe, Phe(X), wherein X may refer to H, Cl, F, methyl group, or any other appropriate modification of Phe. In some embodiments, the tail of the ORL comprises Ppp or NH₂. The present invention is not limited to the aforementioned formula molecules. For reference, DTic refers to D-tetrahydroisoquinoline-3-carboxylic acid.

Table 1 below shows non-limiting examples of ORLs of the present invention. Note that the Phe residues in SEQ ID NO: 10, SEQ ID NO: 11, and SEQ ID NO: 12 are halogenated with F, and the Phe residue in SEQ ID NO: 15 is halogenated with Cl.

TABLE 1
Examples of ORLs

| | | Residue | | | |
|---|---|---|---|---|---|
| SEQ | | (Position from N-terminus to C-terminus) | | | |
| ID NO: | Compound | 1 | 2 | 3 | 4 |
| 7 | LYS729 | Tyr | DAla | Gly | Phe-NH₂ |
| 8 | LYS544 | Tyr | DAla | Gly | Phe-Ppp |
| 9 | LYS540 | Dmt | DAla | Gly | Phe-Ppp |
| 10 | LYS739 | Dmt | DNle | Gly | Phe(4-F)-Ppp |
| 11 | MR106 | Tyr | DNle | Gly | Phe(4-F)-NH₂ |
| 12 | MR107 | Dmt | DNle | Gly | Phe(4-F)-NH₂ |
| 13 | CYF132 | Dmt | DNle | Gly | Phe-NH₂ |
| 14 | LYS644 | Dmt | DNle | Gly | Phe-Ppp |
| 15 | LYS744 | Dmt | DNle | Gly | Phe(4-Cl)-Ppp |
| 16 | LYS702 | Dmt | DTic | | Phe-Ppp |
| 17 | CYF136 | Dmt | DNle | Gly | Phe(4-Cl)-NH₂ |
| 18 | MR119 | Dmt | DNle | Gly | Phe(4-Br)-Ppp |
| 19 | MR111* | Dmt | DNle | Homocys | Phe(4-F)-Ppp |
| | (*see note | | | | |
| | below) | | | | |
| 20 | MR112* | Dmt | Homocys | Gly | Phe(4-F)-Ppp |
| | (*see note | | | | |
| | below) | | | | |
| 21 | MR124 | Dmt | DArg | Phe | Gly-Ppp |
| 22 | MR125 | Dmt | DArg | 1Nal | Gly-Ppp |
| 23 | MR110 | Dmt | DArg | Phe | Lys-Ppp |
| 24 | MR120 | Dmt | DArg | 1Nal | Lys-Ppp |
| 25 | MR122 | Dmt | DArg | | 1Nal-Ppp |
| 26 | MR121 | Dmt | DArg | | Phe(4-Cl)-Ppp |
| 27 | MR114 | Dmt | Pro | Trp | Phe(4-Cl)-Ppp |
| 28 | MR115 | Dmt | Pro | Phe | Phe(4-Cl)-Ppp |
| 29 | MR116 | Dmt | Pro | Phe(4-Cl) | Phe(4-Cl)-Ppp |
| 30 | MR123 | Dmt | Pro | Gly | Phe(4-Cl)-Ppp |

Note MR111 comprises two units of SEQ ID NO; 19, e.g., MR111 comprises (Dmt-DNle-Homocys-Phe(4-F)-Ppp)₂. MR112 comprises two units of SEQ ID NO: 20, e.g., MR112 comprises (Dmt-Homocys-Gly-Phe(4-F)-Ppp)₂.

The ORLs of the present invention may be synthesized as appropriate (see, for example, Lee et al., 2011, J. Med. Chem. 54:382-886). For example, the ORLs of the present invention may be synthesized by a protocol for liquid phase peptide synthesis (LPPS), e.g., using Boc-chemistry in high yields. In some embodiments, halogen modification on the aromatic ring is on the para position, e.g., to help avoid unfavorable steric hindrance.

Table 2 shows analytical data of various multifunctional ORLs of the present invention with a Ppp group at the C-terminus. ^{a}FAB-MS (JEOL HX110 sector instrument) or MALDI-TOF. ^{b}Performed on a Hewlett Packard 1100 [C-18, Vydac, 4.6 mm×250 mm, 5 μm, 10-100% of acetonitrile containing 0.1% TFA within 45 min, 1 mL/min]. ^{c}http://www.vcclab.org/lab/alogps/. ^{d}Low resolution-Mass. ° Retention time. n.d. not determined.

TABLE 2

| ORL/ | | | | | | |
|---|---|---|---|---|---|---|
| SEQ ID | | Molecular | HR MS^{a} (M-TFA + H)⁺ | | HPLC^{b} | |
| NO: | Structure | Formula | observed | calculated | % ACN | ALOGPs^{c} |
| LYS729/7 | Tyr-DAla-Gly-Phe-NH₂ | C₂₃H₂₉N₅O₆ | 456.2239 | 456.2246 | 14.0^{e} | 0.32 |
| MR106/ | Tyr-DNle-Gly-Phe(4-F)-NH₂ | C₂₆H₃₄FN₅O₅ | n.d. | 515.2544 | 39.64 | 1.36 |
| 11 | | | | | | |
| MR107/ | Dmt-DNle-Gly-Phe(4-F)-NH₂ | C₂₈H₃₈FN₅O₆ | n.d. | 543.2857 | 41.39 | 1.75 |
| 12 | | | | | | |
| CYF136/ | Dmt-DNle-Gly-Phe(4-Cl)-NH₂ | C₂₈H₃₈ClN₅O₅ | 560.2653 | 560.2640 | 16.0^{e} | 2.36 |
| 17 | | | | | | |
| CYF132/ | Dmt-DNle-Gly-Phe-NH₂ | C₂₈H₃₉N₅O₅ | 526.3035 | 526.3030 | 16.5 | 1.69 |
| 13 | | | | | | |
| LYS544/8 | Tyr-DAla-Gly-Phe-Ppp | C₃₇H₄₆N₆O₆ | 671.3579 | 671.3557 | 19.1^{e} | 2.80 |
| LYS540/9 | Dmt-DAla-Gly-Phe-Ppp | C₃₉H₅₀N₆O₆ | 699.3852 | 699.3870 | 20.1^{e} | 2.96 |
| LYS644/ | Dmt-DNle-Gly-Phe-Ppp | C₄₂H₅₆N₆O₆ | 741.4325 | 741.4340 | 19.3^{e} | 3.66 |
| 14 | | | | | | |
| LYS739/ | Dmt-DNle-Gly-Phe(F)-Ppp | C₄₂H₅₅FN₆O₆ | 759.4247 | 759.4245 | 20.0^{e} | 3.74 |
| 10 | | | | | | |
| LYS744/ | Dmt-DNle-Gly-Phe(4-Cl)-Ppp | C₄₂H₅₅ClN₆O₆ | 775.3995 | 774.3950 | 53.52 | 4.18 |
| 15 | | | | | | |
| LYS702/ | Dmt-DTic-Phe-Ppp | C₄₄H₅₀ClN₅O₅ | 764.3632 | 765.3658 | 23.0^{e} | 4.91 |
| 16 | | | | | | |
| MR111/ | (Dmt-DNle-Homocys-Phe(4- | C₈₈H₁₁₆F₂N₁₂O₁₂S₂ | 1635.5^{d} | 1635.8324 | 62.24 | 5.58 |
| 19 | F)-Ppp)₂ | | | | | |
| MR112/ | (Dmt-Homocys-Gly-Phe(4-F)- | C₈₀H₁₀₀F₂N₁₂O₁₂S₂ | 1524.4^{d} | 1523.7072 | 56.70 | 4.89 |
| 20 | Ppp)₂ | | | | | |
| MR121/ | Dmt-DArg-Phe(4-Cl)-Ppp | C₄₀H₅₃ClN₈O₅ | 761.3^{d} | 761.3906 | 50.56 | 3.00 |
| 26 | | | | | | |
| MR114/ | Dmt-Pro-Trp-Phe(4-Cl)-Ppp | C₅₀H₅₈ClN₇O₆ | 888.6^{d} | 888.4216 | 62.00 | 5.20 |
| 27 | | | | | | |
| MR116/ | Dmt-Pro-Phe(4-Cl)-Phe(4- | C₄₈H₅₆ClN₆O₆ | 883.37 | 883.3717 | 57.83 | 5.23 |
| 29 | Cl)-Ppp | | | | | |

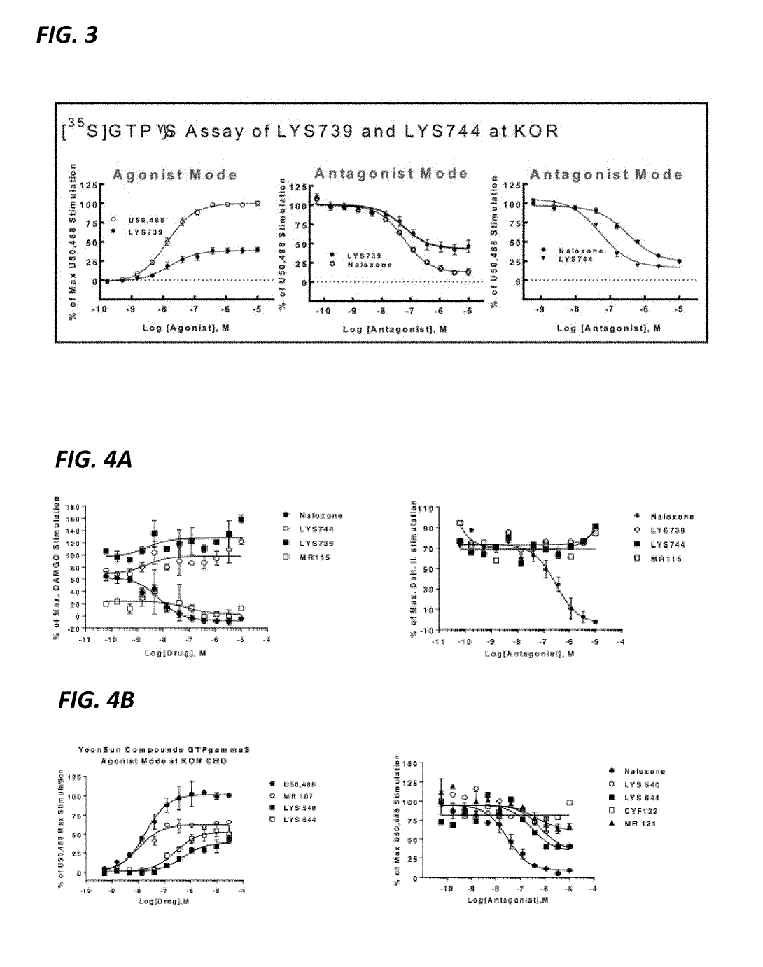

As show n Table 3, Table 4.1, Table 4.2, and FIG. 3 it was surprisingly discovered that the ORL LYS739 (SEQ ID NO: 10, Dmt-DNle-Gly-Phe(4-F)Ppp) interacts with KOR (K=0.70 nM) as well as MOR (Kᵢ=0.02 nM) and DOR (Kᵢ=0.40 nM). Considering well-known structure-activity relationships (SAR) of enkephalin analogues, the sub nanomolar range of binding affinity of LYS739 (SEQ ID NO: 10) at the KOR was unexpected and could not be predicted. LYS739 (SEQ ID NO: 10) turned out to be the first potent MOR/DOR agonist (IC₅₀: 0.26 nM, and 0.37 nM in GPI, and MVD, respectively) and KOR partial agonist/antagonist among ORLs. In GTP-γ-assay, LYS739 (SEQ ID NO: 10) showed mixed partial agonist (EC₅₀=21 nM, Eₘₐₓ=39%)/antagonist activity (EC₅₀=60 nM, Eₘₐₓ=65%) for KOR. Note that in Table 4.1 and Table 4.2, potency and efficacy reported as mean±SEM from each experiment (n=3) independent experiments for both modes; curves use the mean value of each point from each experiment combined together; n.d. is not determined. It was surprisingly discovered that the ORL LYS744 interacts with KOR, as well as MOR and DOR. Interestingly, LYS744, containing a Phe(4-Cl) residue instead of a Phe(4-F), showed full antagonist activity (IC₅₀=52 nM, Iₘₐₓ=122%) in the assay.

TABLE 3
Binding Affinities of Enkephalin Analogs at MOR, DOR, and KOR

| | | Ki (nM) | | |
|---|---|---|---|---|
| SEQ | | MOR | DOR | KOR |
| ID NO: | Compound | [³H]DAMGO | [³H]DPDPE | [³H]Nor-BNI |
| 7 | LYS729 | 2.8 | 300 | 220 |
| 8 | LYS544 | 26 | 5.2 | 190 |
| 9 | LYS540 | 0.38 | 0.36 | n/d |
| 10 | LYS739 | 0.02 | 0.4 | 0.7 |
| 11 | MR106 | n.d | n.d | 210 |
| 12 | MR107 | n.d | n.d | 0.11 |
| 13 | CYF132 | n.d | n.d | 3.4 |
| 14 | LYS644 | 0.39 | 0.18 | n.d |
| 15 | LYS744 | 0.08 | 0.10 | 1.4 |
| 16 | LYS702 | 0.45 | 0.76 | n.d |

TABLE 4.1
Functional Activities of LYS739 (SEQ ID NO: 10) at MOR, DOR, and
KOR

| [³⁵S]GTP-γ-S binding assay | | | | | | |
|---|---|---|---|---|---|---|
| | | | Antagonist | | | |
| Agonist | | | IC₅₀ (nM) | IC₅₀ (nM) | | |
| EC₅₀ (nM) (Eₘₐₓ %) | | | (Iₘₐₓ %) | GPI | MVD | GPI |
| hDOR | rMOR | hKOR | hKOR | (μ) | (δ) | (k) |
| 0.07 (48^{a}) | 0.29 (98^{a}) | 21 (39^{b}) | 60 (65)^{c} | 0.26 | 0.37 | n.d. |

^{a}[total bound-basal]/[basal-nonspecific] × 100.
^{b}Relative % of 10 μM U50,488 stimulation.
^{c}Relative % of naloxone blocking 100 nM U50,488 stimulation,
n/d: not determined.

TABLE 4.2
Functional Activities of LYS744 (SEQ ID NO: 5) at MOR, DOR, and
KOR

| [³⁵S]GTP-γ-S binding assay | | | | | | |
|---|---|---|---|---|---|---|
| | | | Antagonist | | | |
| Agonist | | | IC₅₀ (nM) | IC₅₀ (nM) | | |
| EC₅₀ (nM) (Eₘₐₓ %) | | | (Iₘₐₓ %) | GPI | MVD | GPI |
| hDOR | rMOR [ | hKOR | hKOR | (μ) | (δ) | (k) |
| 0.07 (37^{a}) | 0.14 (58^{a}) | <10 at 10 | 52 (122)^{c} | 1.3 | 1.9 | n.d. |
| | | uM^{b} | | | | |

^{a}[total bound-basal]/[basal-nonspecific] × 100.
^{b}Relative % of 10 μM U50,488 stimulation.
^{c}Relative % of naloxone blocking 100 nM U50,488 stimulation.
n/d: not determined.

Preliminary in vivo studies of LYS739 (SEQ ID NO: 10) showed that intrathecal (i.th.) administration of LYS739 (SEQ ID NO: 10) at 10 μg/5 μl in L₅/L₆ SNL-operated male SD rats can reverse thermal hyperalgesia in nerve injured animals and reverse tactile allodynia. For example, FIG. 4A shows [3S]GTPγS assays: MOR (left) and DOR (right) antagonist modes. LYS739 (SEQ ID NO: 10), LYS744 (SEQ ID NO: 15), and MR115 (SEQ ID NO: 28) do not possess antagonist activity at MOR and DOR. FIG. 4B shows [3S]GTPγS assays: KOR agonist (left) and antagonist (right) modes. LYS540 (SEQ ID NO: 9), LYS644 (SEQ ID NO: 14), and MR121 (SEQ ID NO: 126) are partial agonist/antagonist at KOR. CYF132 (SEQ ID NO: 13) is observed as a partial agonist at KOR. In FIG. 4A, statistical significance was determined by 95% confidence interval (*P<0.05 compared with pre-dose SNL baseline vehicle; #p<0.05 compared with the vehicle at the same time point; n>6). Vehicle was DMSO/Tween 80/Saline (1:1:8). Intravenous (i.v.) administration of LYS739 (SEQ ID NO: 10) (3 mg/mL/Kg) in L₅/L₆ SNL-operated male SD rats shows reversal of thermal hyperalgesia. This represents high potency of analgesic effects through MOR (and DOR).

For reference, Table 5 lists examples of ORLs with various tail portions (e.g., NH₂ and Tail Compounds 1-5). Structures of the Tails (e.g., anilidopiperidine moieties) can be found in FIG. 2. Table 5 also shows lipophilicity values and MOR/DOR agonist activities of the ORLs. Note that SEQ ID NO: 8 refers to both LYS544 and LYS436.

TABLE 5
MOR/DOR agonist activities of C-terminal modified lipophilic
enkephalin analogues

| | | | Kᵢ (nM) |
|---|---|---|---|
| ORL | Tail | aLogP | MOR/DOR |
| LYS729 | —NH₂ | 0.32 | 2.8/300 |
| Tyr-DAla-Gly-Phe-NH₂ | | | |
| (SEQ ID NO: 7) | | | |
| LYS416 | 4-Anilidopiperidine | 2.93 | 14/14 |
| Tyr-DAla-Gly-Phe-Tail | analogue 1 | | |
| (SEQ ID NO: 31) | | | |
| LYS620 | 4-Anilidopiperidine | 2.63 | 1.2/3.7 |
| Tyr-DAla-Gly-Phe-Tail | analogue 2 | | |
| (SEQ ID NO: 32) | | | |
| LYS429 | 4-Anilidopiperidine | 4.04 | 1.1/6.1 |
| Tyr-DAla-Gly-Phe-Tail | analogue 3 | | |
| (SEQ ID NO: 33) | | | |
| LYS544 (or LYS436) | 4-Anilidopiperidine | 2.80 | 23/0.69 |
| Tyr-DAla-Gly-Phe-Ppp | analogue 4 (Ppp) | | |
| (SEQ ID NO: 8) | | | |
| LYS437 | 4-Anilidopiperidine | 2.13 | 5.7/3.2 |
| Tyr-DAla-Gly-Phe-Tail | analogue 5 | | |
| (SEQ ID NO: 34) | | | |

The present invention also features ORLs that are derived from LYS739 (SEQ ID NO: 10), e.g., LYS739 analogs. In some embodiments, the ORLs are obtained by modifying LYS739 (SEQ ID NO: 10) by substitution, dimerization, and/or cyclization. Modifications may involve the incorporation of an unnatural amino acid and/or constrained amino acids. For example, in some embodiments, Dmt is substituted with trimethyltyrosine (Tmt). In some embodiments, the ORL comprises 2-methyl-3-(2′,6′-dimethyl-4′-hydroxyphenyl)-propionic acid (Mdp).

In some embodiments, the ORL comprises a bivalent ligand. In some embodiments, a disulfide bond is used to link two monomeric pharmacophores. For example, a disulfide bond may be used through a homocysteine residue at position 2 (or 3). In some embodiments, ORLs comprise cyclic structures, e.g., the ORLs are cyclic and retain the pharmacophoric structure for the receptors within a constrained structure, e.g., since linear peptide ligands can be flexible even with multiple modifications due to high flexibility of enkephalins. Cyclization may be through the formation of various bonds such as a disulfide and a lactam, but is not limited to these mechanisms.

In some embodiments, the ORLs are bifunctional ligands. In some embodiments, the ORLs are trifunctional ligands. In some embodiments, ORLs are constructing based on an enkephalin tetrapeptide (Tyr-Gly-Gly-Phe-NH2, SEQ ID NO: 46). In some embodiments, ORLs are constructed using endomorphin-1 (EM-1) and/or DALDA (D-Arg², Lys⁴]dermorphin. The present invention features ORL designs using EM-1 (Tyr-Pro-Trp-Phe-NH₂, SEQ ID NO: 35) and DALDA (Tyr-DArg-Phe-Lys-NH₂, SEQ ID NO: 36). The present invention also features ORLs using endomorphin-2 (EM-2) (Tyr-DArg-Phe-Lys-NH2, SEQ ID NO: 45).

Various ORLs (e.g., analogs of LYS739 (SEQ ID NO: 10)) were tested for their binding affinities at MOR, DOR, and KOR using [³H]-Diprenorphine in the membranes of Chinese Hamster Ovary (CHO) cells expressing the relevant human opioid receptor. Analogues with particular binding affinity (Ki<10 nM for MOR and DOR; Ki<30 nM for KOR) as well as others were tested for receptor functional activity in the [3S]-GTPγS assay. In this assay, antagonist activity at all three receptors expressed in CHO cells were determined by the inhibition of stimulation caused by 100 nM of control agonist (DAMGO for MOR, SNC80 for DOR, U50,488 for KOR) in a 96-well plate. Table 6 summarized in vitro biological activities of multifunctional ligands at MOR, DOR, and KOR with a Ppp group at the C-terminus. (Note: ^{a}=Competition analyses were carried out using membrane preparations from transfected HNB9.10 cells that constitutively expressed the respective receptor types; ^{b}=[³H]DAMGO, K_{d}=0.85 nM; ^{c}=[³H]DPDPE, Kd=0.50 nM; ^{d}=[³H]U69,593, Kd=5.3 nM; ^{e}=Expressed in CHO cells; ^{f}=Mean±SEM of the % relative to 10 μM U50,488 stimulation; ^{g}=Mean±SEM of the % relative to 10 μM naloxone inhibition of 100 nM U50,488; ^{h}=at 10 μM.

TABLE 6

| | | | | | KOR^{e} | | | | |
|---|---|---|---|---|---|---|---|---|---|
| SEQ | | | | | [³⁵S]GTPγS-binding | | | | |
| ID | | Kᵢ, nM^{a} | | | EC₅₀, | Eₘₐₓ, | IC₅₀, | Iₘₐₓ, | |
| NO: | ligand | MOR^{b} | DOR^{c} | KOR^{d} | nM | %^{f} | nM | %^{g} | KOR function |
| 7 | LYS729 | 2.8 | 300 | 2000 | — | <30^{h} | — | <10^{h} | no function |
| 11 | MR106 | 2.3 | 0.44 | 7.8 | — | 70^{h} | | | weak agonist |
| 12 | MR107 | 4.5 | 0.99 | 604 | 10 | 62 | 250 | 37 | partial |
| | | | | | | | | | agonist/antagonist |
| 17 | CYF136 | 2.9 | 0.61 | 156 | 7.0 | 18 | 66 | 60 | partial |
| | | | | | | | | | agonist/antagonist |
| 13 | CYF132 | 1.1 | 0.25 | 3.4 | 84 | 59 | n.c.* | — | partial agonist |
| 8 | LYS544 | 23 | 0.69 | 2000 | — | <10^{h} | — | <10^{h} | no function |
| 9 | LYS540 | 0.38 | 0.36 | 21 | 540 | 40 | 630 | 49 | partial |
| | | | | | | | | | agonist/antagonist |
| 14 | LYS644 | 0.39 | 0.18 | 77 | 260 | 53 | 290* | 70 | partial |
| | | | | | | | | | agonist/antagonist |
| 10 | LYS739 | 0.02 | 0.40 | 0.70 | 21 | 39 | 60 | 65 | partial |
| | | | | | | | | | agonist/antagonist |
| 15 | LYS744 | 0.10 | 0.08 | 1.4 | — | <10%^{h} | 52 | 122 | antagonist |
| 16 | LYS702 | 0.45 | 0.76 | 2000 | — | <10%^{h} | | | no function |
| 19 | MR111 | 0.02 | 2.6 | 220 | | | | | MOR selective |
| 20 | MR112 | n.c. | 9.9 | n.c. | | | | | DOR selective |
| 26 | MR121 | 1100 | 960 | 62 | 470 | 32 | 450* | 38 | partial |
| | | | | | | | | | agonist/antagonist |
| 27 | MR114 | 4700 | 68 | 200 | | | | | DOR selective |
| 29 | MR116 | 990 | 30 | n.c. | | | | | DOR selective |

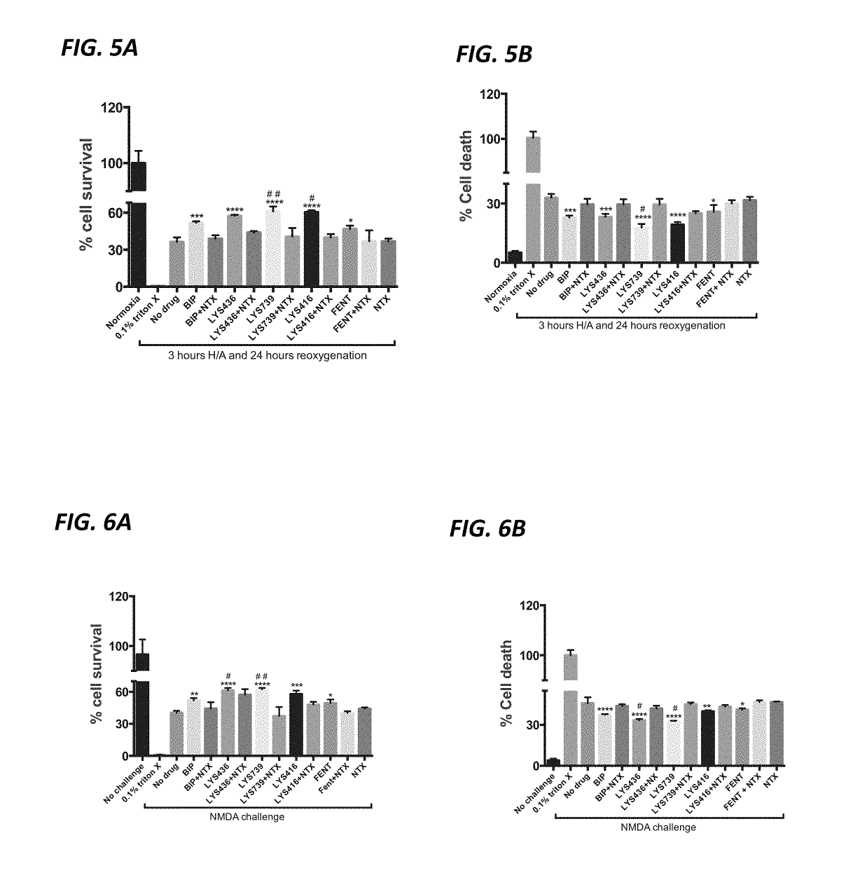

Analogues were tested for their activity at KOR, and GTPγS assays were performed at the MOR and DOR for LYS739 (SEQ ID NO: 10) and LYS744 (SEQ ID NO: 15) (see FIG. 4A). The assay results shows that two ligands are pure agonists for the MOR and DOR. GTPγS assays showed that LYS540 (SEQ ID NO: 9) and LYS644 (SEQ ID NO:14) are partial agonist/antagonist for the KOR, which has a potential to reduce KOR related side effects (see FIG. 5B). MR107 (SEQ ID NO: 12) and CYF136 (SEQ ID NO: 17) also revealed partial agonist/antagonist activities.

The present invention also features ORLs having half lives longer than 4 hours. For example, in some embodiments, the ORL has a half life longer than 1 hour. In some embodiments, the ORL has a half life longer than 2 hours. In some embodiments, the ORL has a half life longer than 3 hours. In some embodiments, the ORL has a half life longer than 4 hours. In some embodiments, the ORL has a half life longer than 5 hours. In some embodiments, the ORL has a half life longer than 10 hours. In some embodiments, the ORL has a half life longer greater than 24 hours.

In some embodiments, the ORL is 4 amino acids in length. In some embodiments, the ORL is 5 amino acids in length. In some embodiments, the ORL is 6 amino acids in length. In some embodiments, the ORL is 7 amino acids in length. In some embodiments, the ORL is 8 amino acids in length. In some embodiments, the ORL is 9 amino acids in length. In some embodiments, the ORL is 10 amino acids in length. In some embodiments, the ORL is more than 10 amino acids in length.

In some embodiments, the ORL is between 4 to 6 amino acids in length. In some embodiments, the ORL is between 4 to 7 amino acids in length. In some embodiments, the ORL is between 4 to 8 amino acids in length. In some embodiments, the ORL is between 4 to 9 amino acids in length. In some embodiments, the ORL is between 4 to 10 amino acids in length. In some embodiments, the ORL is between 4 to 20 amino acids in length. In some embodiments, the ORL is between 4 to 30 amino acids in length. In some embodiments, the ORL is between 4 to 40 amino acids in length. In some embodiments, the ORL is between 4 to 50 amino acids in length.

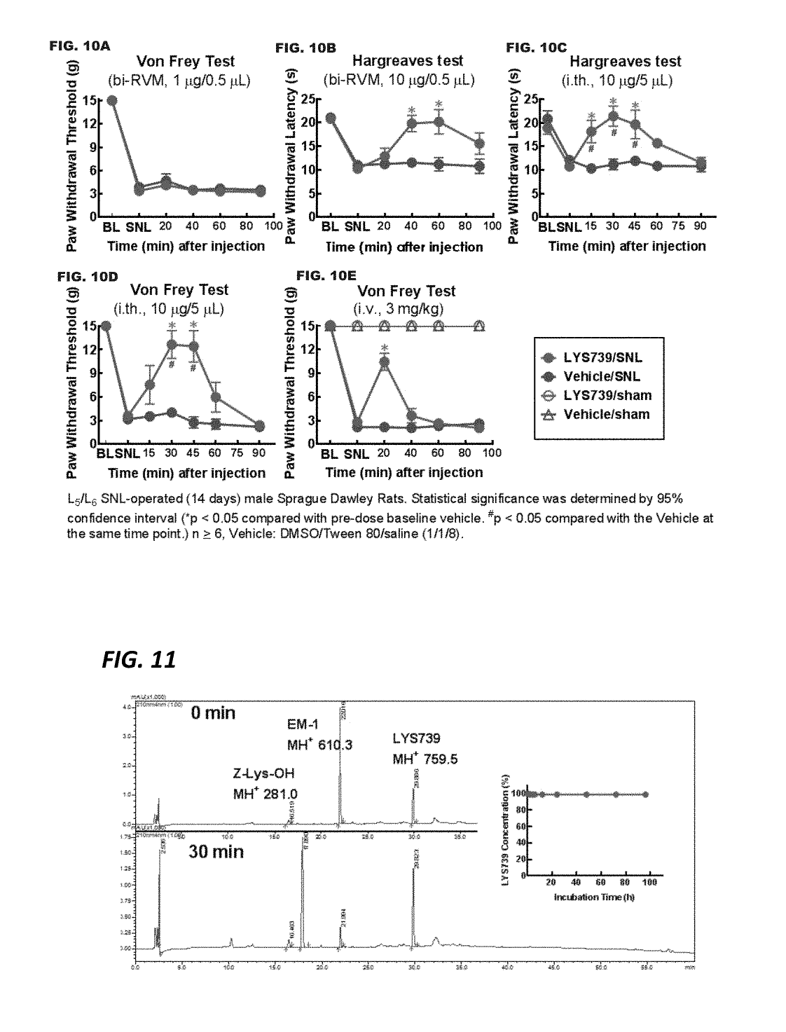

As shown in FIG. 10A-10E, bilateral RVM or intrathecal (i.th.) injections of LYS739 (10 μg/0.5 μL) significantly reversed tactile allodynia and thermal hyperalgesia in the Hargreaves test and the von Frey test, respectively, using L₅/L₆ SNL-operated male Sprague Dawley (SD) rats. A relatively low dose of intravenous (i.v.) (3 mg/kg) LYS739 also significantly attenuated nerve injury induced tactile allodynia in the rats. The peak time of antiallodynic effect of LYS739 was observed 20 min post-administration, with a mean paw withdrawal threshold significantly higher than that of vehicle-treated injury. LYS739 is considered to possess great potential in having both potent and efficacious analgesia after systemic administration and is capable of crossing the BBB (comparing i.t. with i.v. administration). Efforts to improve the biological activity of enkephalin also increased metabolic stability due to the three non-natural amino acid modifications. LYS739 was very stable in human plasma. No degradation was observed after 96 h incubation at 37° C., while EM-1, which was used to validate the plasma's activity as a reference compound, was degraded very quickly in an hour (FIG. 11).

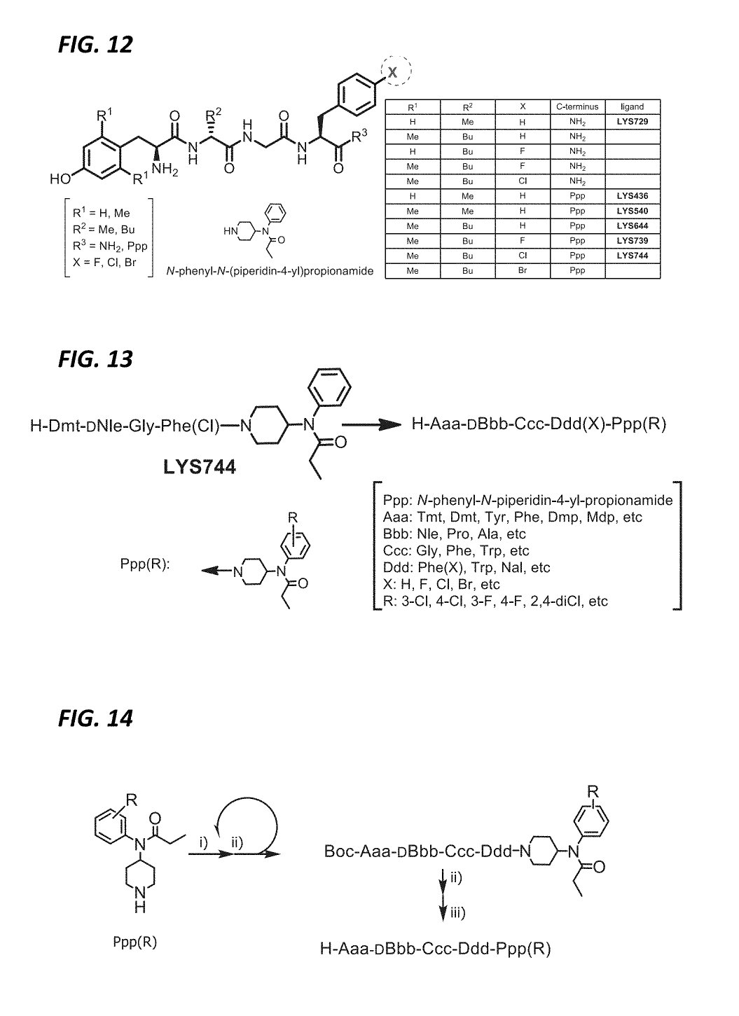

Examples of other enkephalin analogues may include but are not limited to those shown in FIG. 12. FIG. 13 shows an example of the design of multifunctional opioid ligands with MOR/DOR agonist and KOR antagonist activity. As an example, the Ppp(R) group may be retained at the C-terminus for these modifications. In some embodiments, Tyr residue may be replaced with a Dmt residue or a α-methyl-2,6-dimethyltyrosine (Tmt) residue, which is more sterically hindered due to an extra methyl group. In some embodiments, 2-methyl-3-(2,6-dimethyl-4-hydroxyphenyl)propanoic acid (Mdp), which has been known to reverse KOR activity dramatically from agonist to antagonist, may be used to investigate the reversal or enhancement of KOR activity and selectivity. This modification is also known to delete MOR/DOR agonist activity, and therefore may result in the discovery of a highly selective enkephalin analogue with antagonist activity at KOR. The Phe residue in ligands may be substituted with Phe(p-X) for altering receptor selectivity and inducing KOR interaction. The modifications at position 4 with a halogen may create novel analogues showing diverse KOR activities. Halogen modification on the aromatic ring may be limited to the para position, e.g., due to unfavorable steric hindrance effects that can lead to suboptimal binding interactions.

FIG. 14 shows a scheme for the synthesis of multifunctional opioid analogs. For example, the present invention may feature a protocol for liquid phase peptide synthesis (LPPS) using Boc-chemistry, which allows synthesis of peptides on the multi gram-scale through a robust procedure. This method may benefit from unnecessary C-terminal masking and unmasking steps due to a PPP(R) group at the C-terminus, and unnecessary side group protection. Commercially available PPP(R) may be used for the synthesis. During synthesis, intermediate peptides may be easily isolated by a simple precipitation using diethyl ether, which may avoid tedious purification steps. The simple isolation of intermediate peptides may allow short synthetic steps with high purity (ex. ≥98% for LYS744) in good overall yields (ex. ≥40% for LYS744). Compounds synthesized may be characterized by RP-HPLC (Hewlett Packard 1100, C-18, Vydac, 4.6 mm×250 mm, 5 μm, 10-90% of acetonitrile containing 0.1% TFA within 40 min, 1 mL/min.), HR-MS (Brucker 9.4 T Apex-Qh FTICR, JEOL HX110 sector instrument, or Brucker Ultraflex III MALD TOF-TOF), and NMR (Brucker DRX-600).

In some embodiments, competitive radioligand binding assays and cell based functional assays are performed. In some embodiments, compounds with a binding affinity of about Kᵢ<100 nM for MOR, DOR and KOR may be tested for receptor functional activity in a cyclic AMP assay. Compounds that show partial agonist (EC₅₀<100 nM, Eₘₐₓ<40%) or antagonist activity (IC₅₀<100 nM, Iₘₐₓ>60%) at the KOR and agonist activity (EC₅₀<100 nM, Eₘₐₓ>70%) at the MOR and DOR in the cyclic AMP assay may be used for off-target screening. In some embodiments, binding affinity (K) will be determined by radioligand competition analysis using [³H]Diprenorphine for MOR, DOR, and KOR, in cell membrane preparations from stably transfected CHO cells expressing respective receptor types.

In some embodiments, cAMP accumulation may be measured. As a non-limiting example, in some embodiments, MOR, DOR, and KOR-CHO cells as above may be plated in 96 well culture microplates, and recovered overnight. The cells may then be serum starved for 20 minutes in serum free medium with 500 μM IBMX, followed by 15 minutes of treatment with 500 μM IBMX, 100 μM forskolin, and concentration curves of experimental drug or reference agonist (DAMGO for MOR, SNC80 for DOR, U50,488 for KOR). Antagonist measurements may be performed using a concentration curve of experimental drug or reference antagonist combined with a fixed concentration of agonist (EC₉₀). The incubation may be terminated, and lysates may be combined with ˜1 pmol of [³H]cAMP and 7 μg of recombinant PKA, and incubated for 1 hour at room temperature. The reaction may be harvested and analyzed to generate potency (EC₅₀/IC₅₀) and efficacy (Eₘₐₓ/I_{Max}) values for each compound. In some embodiments, off-target activities of compounds selected from in vitro analysis may be confirmed by the screening offered by the National Institute of Mental Health's Psychoactive Drug Screening Program (contract # HHSN-271-2008-025C (51). Note LYS739 did not show any off-target activities. In some embodiments, compounds with binding affinity below 100-fold vs. MOR/DOR/KOR for the other off-target receptors may be excluded from further studies.

In some embodiments, NMR analysis and/or computer modeling experiments are used to help identify structural features of enkephalin that may be important for KOR antagonist activity.

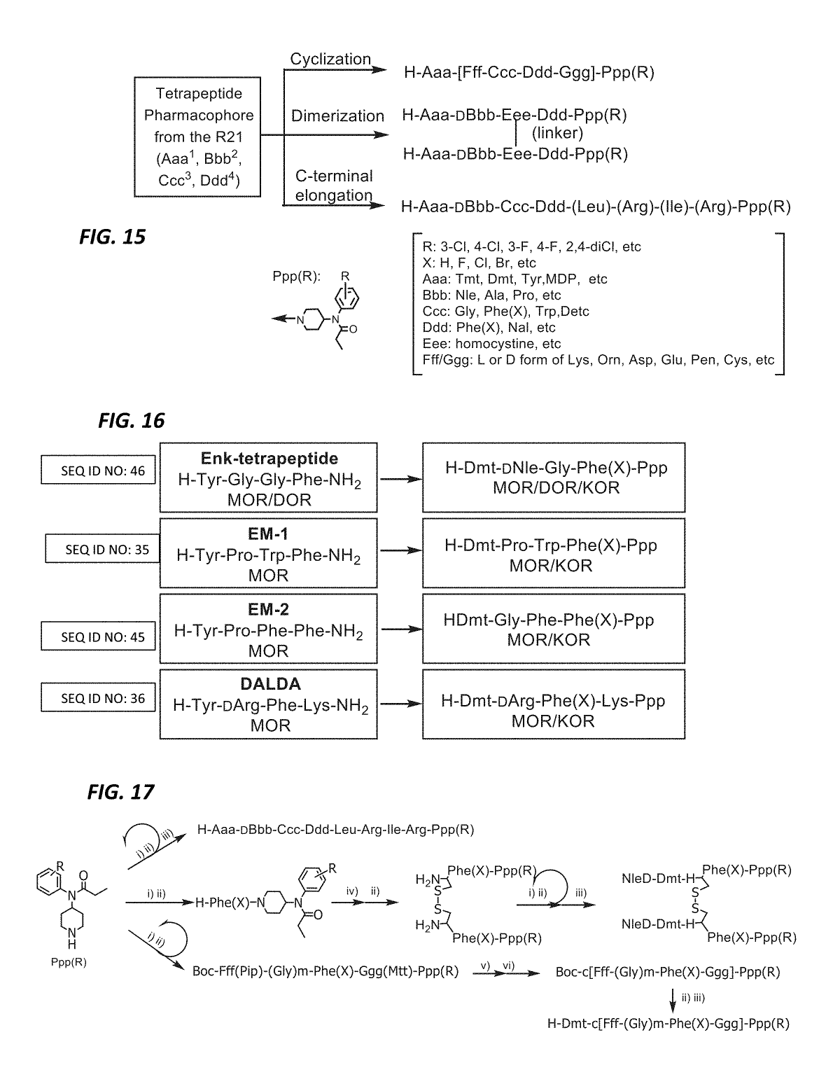

FIG. 15 shows a non-limiting example of design of multifunctional ligands. The multifunctional ligands may be synthesized with high efficacy and high potential bioavailability. Modifications may include i) cyclization, ii) dimerization, and iii) C-terminal elongation. In some embodiments, cyclic ligands that retain the pharmacophore structure for the receptors within a constrained ring structure may be synthesized (in some embodiments, cyclic peptides may possess high potential to increase both biological activity and bioavailability due to their conformational rigidity).

Cyclization may be achieved through the formation of various bonds such as a disulfide and a lactam bond. Bivalent ligands may be built on the pharmacophore structure. In order to link two monomeric pharmacophores, a disulfide bond may be utilized, e.g., through a homocysteine residue at position 2 (or 3). In some embodiments, the C-terminal chain elongation may be applied to enhance the KOR activity. For example, this modification may feature attachment of Leu⁵, Arg⁶, Ile⁸, and Arg⁹ residues in the dynorphin structure to a tetrapeptide scaffold.

The present invention also provides modifications of several known opioid ligands, such as endomorphin-1 (EM-1) (Kᵢ=0.36 nM for MOR with 4,000- and 15,000-fold preference over DOR and KOR, respectively) (70) and [D-Arg², Lys⁴]-dermorphin (DALDA) (Ki=1.69 nM for MOR with 11,000- and 2,500-fold preference over DOR and KOR, respectively) (see FIG. 16).

For example, in some embodiments, the ORL is derived from DALDA, e.g., according to Formula 5: Aaa-DArg-Ccc-Ddd-Eee. In some embodiments, Aaa is selected from Tyr or 2′-6′-dimethyltyrosine (Dmt); Ccc is selected from Phe, Phe(X), or 1-naphthylalanine (1Nal); Ddd is selected from Lys, Gly or is absent; Eee is a 4-anilidopiperidine moiety (e.g., Ppp); and X is selected from F, Cl, or Br. In some embodiments, Ppp comprises Ppp(R), wherein R comprises a halogen. In some embodiments, R is selected from 3-Cl, 4-Cl, 3-F, 4-F, and 2,4-diCl. In some embodiments, the ORL is SEQ ID NO: 21, SEQ ID NO: 22, SEQ ID NO: 23, SEQ ID NO; 24, SEQ ID NO: 25, SEQ ID NO: 26, SEQ ID NO: 42, or SEQ ID NO: 43.

In some embodiments, the ORL is derived from EM-1 or EM-2, e.g., the ORL is according to Formula 6: Aaa-Pro-Cco-Phe(X)Eee. In some embodiments, Aaa is selected from Tyr or 2′-6′-dimethyltyrosine (Dmt); Ccc is selected from Trp, Phe, Gly, or Phe(X); Eee is a 4-anilidopiperidine moiety (e.g., Ppp), and X is selected from F, Cl, or Br. In some embodiments, Ppp comprises Ppp(R), wherein R comprises a halogen. In some embodiments, R is selected from 3-Cl, 4-Cl, 3-F, 4-F, and 2,4-diCl. In some embodiments, the ORL is selected from SEQ ID NO: 27, SEQ ID NO: 28, SEQ ID NO: 29, or SEQ ID NO: 30.

In some embodiments, the ORL is derived from FE20066, e.g., according to Formula 7: DPhe-DPhe-DNle-Ddd-Eee. In some embodiments, Ddd is selected from D-Arg or D-Lys, and Eee is a 4-anilidopiperidine moiety (e.g., Ppp). For example, in some embodiments, Ddd is D-Arg and Eee is Ppp. In some embodiments, Ddd is D-Lys and Eee is Ppp. In some embodiments, Ppp comprises Ppp(R), wherein R comprises a halogen. For example, in some embodiments, Ddd is D-Arg and Eee is Ppp(R), wherein R comprises a halogen (e.g., Cl, F, Br). In some embodiments, Ddd is D-Lys and Eee is Ppp(R), wherein R comprises a halogen (e.g., Cl, F, Br). In some embodiments, R is selected from 3-Cl, 4-Cl, 3-F, 4-F, and 2,4-diCl. In some embodiments, the ORL is according to SEQ ID NO: 44.

The present invention also provides an opioid receptor ligand dimer according to SEQ ID NO: 19. The present invention also provides an opioid receptor ligand dimer according to SEQ ID NO: 20.

FIG. 17 shows a non-limiting example of synthesis of compounds. In some embodiments, synthesis features LPPS using standard Boc-chemistry. For the synthesis of cyclic compounds, highly acid labile protecting groups may be used. In some embodiments, after chain elongation, linear compounds may be cyclized in highly diluted solution to avoid intermolecular dimerization.

In some embodiments, modifications to the peptide ligand (e.g., incorporation of a Ppp group at the C-terminus) enhances metabolic stability and/or lipophilicity and/or blood brain barrier (BBB)/central nervous system (CNS) permeability.

Table 7 shows analytical data for several multifunctional opioid receptor ligands (ORLs) with a Ppp group at the C-terminus. For reference: ^{a}FAB-MS (JEOL HX110 sector instrument) or MALDI-TOF. ^{b}Performed on a Hewlett Packard 1100 [C-18, Vydac, 4.6 mm×250 mm, 5 μm, 10-100% of acetonitrile containing 0.1% TFA within 45 min, 1 mL/min]. ^{c}http://www.vcclab.org/lab/alogps/. ^{d}Low resolution-Mass. n.d. not determined.

TABLE 7

| | | | | | HPLC^{b} | |
|---|---|---|---|---|---|---|
| | | molecular | HR MS^{a} (M-TFA + H)⁺ | | Retention | |
| analogues | structure | formula | observed | calculated | Time | ALOGPs^{c} |
| MR119 | Dmt-DNle-Gly- | C₄₂H₅₅BrN₆O₆ | 821.34156 | 819.3445 | 25.4 | 4.25 |
| SEQ ID | Phe(4-Br)-Ppp | | | | | |
| NO: 18 | | | | | | |
| CYF202 | Dmt-DNle-Gly- | C₄₈H₆₆ClN₇O₇ | 872.51049 | 872.50805 | 23.8 | 4.09 |
| SEQ ID | Phe(4-F)-Leu- | | | | | |
| NO: 38 | Ppp | | | | | |
| MR127 | Dmt-Tic-Gly- | C₄₆H₅₄N₆O₆ | 787.41646 | 787.4184 | 24.0 | |
| SEQ ID | Phe-Ppp | | | | | |
| NO: 39 | | | | | | |
| MR128 | Dmt-Tic-Gly- | C₄₆H₅₃FN₆O₆ | 805.40757 | 805.4090 | 24.4 | |
| SEQ ID | Phe(4-F)-Ppp | | | | | |
| NO: 40 | | | | | | |
| MR129 | Dmt-Tic-Gly- | C₄₆H₅₃ClN₆O₆ | 821.37849 | 821.3795 | 26.1 | |
| SEQ ID | Phe(4-Cl)-Ppp | | | | | |
| NO: 41 | | | | | | |
| MR111 | (Dmt-DNle- | C₈₈H₁₁₆F₂N₁₂O₁₂S₂ | 1635.5^{d} | 1635.8324 | 26.1 | 5.58 |
| SEQ ID | Homocys-Phe(4- | | | | | |
| NO: 19 | F)-Ppp), | | | | | |
| MR112 | (Dmt-Homocys- | C₈₀H₁₀₀F₂N₁₂O₁₂S₂ | 1524.4^{d} | 1523.7072 | 23.4 | 4.89 |
| SEQ ID | Gly-Phe(4-F)- | | | | | |
| NO: 20 | Ppp), | | | | | |
| MR110 | Dmt-DArg-Phe- | C₄₆H₆₆N₁₀O₆ | 855.5^{d} | 855.5246 | 13.9 | 1.89 |
| SEQ ID | Lys-Ppp | | | | | |
| NO: 23 | | | | | | |
| MR232 | Dmt-DArg- | C₄₆H₆₅ClN₁₀O₆ | 889.48402 | 889.4856 | 17.0 | |
| SEQ ID | Phe(4-Cl)-Lys- | | | | | |
| NO: 42 | Ppp | | | | | |
| MR233 | Dmt-DArg- | C₄₆H₆₅FN₁₀O₆ | 873.51465 | 873.5152 | 16.2 | |
| SEQ ID | Phe(4-F)-Lys- | | | | | |
| NO: 43 | Ppp | | | | | |
| MR124 | Dmt-DArg-Phe- | C₄₂H₅₇N₉O₆ | 784.4^{d} | 784.4511 | 16.6 | 1.92 |
| SEQ ID | Gly-Ppp | | | | | |
| NO: 21 | | | | | | |
| MR125 | Dmt-DArg-1Nal- | C₄₆H₅₉N₅O₆ | 834.4661 | 834.4667 | 19.9 | 2.83 |
| SEQ ID | Gly-Ppp | | | | | |
| NO: 22 | | | | | | |
| MR120 | Dmt-DArg-1Nal- | C₅₀H₆₈N₁₀O₆ | 905.5^{d} | 905.5402 | 17.4 | 2.67 |
| SEQ ID | Lys-Ppp | | | | | |
| NO: 24 | | | | | | |
| MR122 | Dmt-DArg-1Nal- | C₄₄H₅₆N₈O₅ | 777.5^{d} | 777.4453 | 21.4 | 3.25 |
| SEQ ID | Ppp | | | | | |
| NO: 25 | | | | | | |
| MR121 | Dmt-DArg- | C₄₀H₅₃ClN₈O₅ | 761.3^{d} | 761.3906 | 20.3 | 3.00 |
| SEQ ID | Phe(4-Cl)-Ppp | | | | | |
| NO: 26 | | | | | | |
| MR114 | Dmt-Pro-Trp- | C₅₀H₅₈ClN₇O₆ | 888.6^{d} | 888.4216 | 26.0 | 5.20 |
| SEQ ID | Phe(4-Cl)-Ppp | | | | | |
| NO: 27 | | | | | | |
| MR115 | Dmt-Pro-Phe- | C₄₈H₅₇ClN₆O₆ | 849.41 | 849.4107 | 22.5 | 4.78 |
| SEQ ID | Phe(4-Cl)-Ppp | | | | | |
| NO: 28 | | | | | | |
| MR116 | Dmt-Pro-Phe(4- | C₄₈H₅₆ClN₆O₆ | 883.37 | 883.3717 | 23.9 | 5.23 |
| SEQ ID | Cl)-Phe(4-Cl)- | | | | | |
| NO: 29 | Ppp | | | | | |
| MR123 | Dmt-Pro-Gly- | C₄₁H₅₁ClN₆O₆ | 759.2^{d} | 759.3636 | 23.0 | 3.68 |
| SEQ ID | Phe(4-Cl)-Ppp | | | | | |
| NO: 30 | | | | | | |
| MR231 | DPhe-DPhe- | C₄₄H₆₁N₇O₅ | 768.48061 | 768.4814 | 19.9 | |
| SEQ ID | DNle-DLys-Ppp | | | | | |
| NO: 44 | | | | | | |

### Example 1

Example 1 describes non-limiting approaches to designing ORLs.

Step 1: Discover pharmacophoric structures of EM-1 and DALDA for MOR agonist/KOR antagonist activities. The C-terminus of EM-1 and DALDA may be modified with Ppp(R) (the R group may be decided by SAR results). This modification may improve their lipophilicities (aLogP increase >2) and metabolic stabilities, and thus afford high potential of BBB penetration. This modification may cause a biological profile change. The Ppp(R) group may be kept at the C-terminus, and the other positions may be modified. Substitution of Tyr with 2′,6′-dimethyltyrosine (Dmt) in opioid peptides can increase opioid activities dramatically, thus a Tyr¹ residue may be replaced in both ligands with a Dmt residue or a β-methyl-2,6-dimethyltyrosine (Tmt) residue, which is more sterically hindered due to an extra methyl group. EM-1 and DALDA have distinct primary structures in positions 2, 3, and 4 but a Phe residue in common. The Phe residue in both ligands may be substituted with Phe(p-X) for altering receptor selectivity and inducing KOR interactions. A Phe³ residue in DALDA may also be substituted with a Phe(p-X) residue to observe SAR. However, to conserve its MOR selectivity over DOR, positions 2 and 4 of DALDA may be limited to basic amino acid residues. Likewise, position 2 of EM-1 may be limited to turn making amino acid residue. A Trp³ residue in EM-1 may be modified with other aromatic amino acid residues.

Step 2: Build dimerized ligands of MOR agonist/KOR antagonist using pharmacophores discovered in the first step. Position 2 and 4 of EM-1 and DALDA, respectively, may be consumed. Two homo pharmacophores may be linked through a disulfide bond of homocysteine residue. Cyclic bifunctional ligands may be designed. Insertion (l, m, and/or n=1) or deletion (l, m, and/or n=0) of Bbb, Ccc, and Ddd may optimize the distance between two aromatic rings, which may be the most important factor of high potency and selectivity.

### Example 2—Multifunctional ORLs as Neuroprotectants for Ischemic Stroke Treatment

Ischemic stroke is one of the leading causes of mortality and morbidity in the world. Example 2 describes the evaluation of multifunctional ORLs, e.g., LYS436_(SEQ ID NO: 8), LYS739 (SEQ ID NO: 10) and LYS416 (YGGF-Ppp, SEQ ID NO: 37), for their neuroprotective potential using in vitro and in vivo ischemic models. In vitro, neuronal death and total reactive oxygen species level, upon exposure to hypoxia-aglycemia followed by reoxygenation or challenged with NMDA was significantly decreased when treated with non-selective opioid agonists compared to no drug treatment group. Fluorinated enkephalin-fentanyl conjugate, LYS739 (SEQ ID NO: 10) showed better neuroprotection in all in vitro ischemic models compared to biphalin. An in vivo mouse middle cerebral artery occlusion (MCAO) stroke model was utilized to screen biphalin and LYS739 (SEQ ID NO: 10). Both agonists significantly decreased brain infarct ratio and edema ration measured with TTC staining compared to saline treated group. Neuronal deficit was improved in terms of neurological score and locomotor activity with LYS739 (SEQ ID NO: 10) and biphalin treatment. All enkephalin fentanyl conjugates and biphalin demonstrated better neuroprotection compared to fentanyl treated groups. Neuroprotective effects of biphalin and multivalent analogs were reversed, in most cases, by naltrexone, a non-selective opioid antagonist. This suggests that LYS739 (SEQ ID NO: 10) is a potential neuroprotective agent for ischemic stroke.

Primary cortical neuron survival upon exposure to 3 hr H/A and 24 hr reperfusion in presence or absence of fentanyl analogs and biphalin (10 nM) was determined using MTT (see FIG. 5A) and LDH (see FIG. 5B) assays. In MTT assay, fentanyl analogs, LYS436 (57.9% more neuronal survival, p<0.0001), LYS739 (68.1% more neuronal survival, p<0.0001) and LYS416 (66.4% more neuronal survival, p<0.0001) and biphalin (42.6% more neuronal survival, p<0.001) and fentanyl (28.7% more neuronal survival, p<0.05) reproducibly improved neuronal survival compared to no drug treatment group. The protective effect of fentanyl analogs, LYS436 (p>0.05), LYS739 (p<0.01) and LYS416 (p<0.05) were significantly better than that of biphalin. They also showed better neuroprotection (LYS436: p<0.05, LYS739: p<0.0001 and LYS416: p<0.001) compared to fentanyl itself. Among the analogs, LYS739 showed the most significant activity in terms of neuronal survival. Likewise, LDH assay showed reproducible, statistically significant neuroprotection upon treatment with biphalin (30.5% less LDH release, p<0.001), LYS436 (29.37% less LDH release, p<0.001), LYS739 (45.7% less LDH release, p<0.0001), LYS416 (41.28% less LDH release, p<0.0001) and FENT (21.59% less LDH release, p<0.05) compared to no drug treated group. In comparison to biphalin, LYS739 (p<0.05) showed less neuronal death upon H/A and reoxygenation exposure. The fentanyl analogs showed less neuronal death compared to fentanyl itself. Notably, non-selective OR antagonist NTX reversed the effect of biphalin and fentanyl analogs in both assays. No statistical significant difference was found for NTX treated group compared to no drug treated group in both assays.

The effect of three fentanyl analogs, LYS436, LYS739 and LYS416 and biphalin and fentanyl (10 nM) were evaluated in primary cortical neurons exposed to 50 μM NMDA for 3 hours followed by 24 hours normal condition media exposure. Relative neuronal survival and cytotoxicity were quantified using MTT (see FIG. 6A) and LDH (see FIG. 6B) assay, respectively. MTT assay showed that LYS436 (52.1% more neuronal survival, p<0.0001), LYS739 (54.7% more neuronal survival, p<0.0001), LYS416 (43.4% more neuronal survival, p<0.001), biphalin (28.7% more neuronal survival, p<0.01) and fentanyl (22.6% more neuronal survival, p<0.05), which was statistically significant when compared to no drug treated group. Compared to biphalin, fentanyl analog LYS436 (p<0.05) and LYS739 (p<0.01) showed better neuroprotection in terms of neuronal survival quantified with MTT assay kit. These two analogs, LYS436 (p<0.01) and LYS739 (p<0.001) also increased neuronal survival when compared to fentanyl itself. Similar reproducible results were observed when the neuroprotective effect was evaluated with an LDH assay kit. With LDH assay, LYS436 (27.5% less LDH release, p<0.0001), LYS739 (28.6% less LDH release, p<0.0001), LYS416 (12.9% less LDH release, p<0.01), biphalin (18.4% less LDH release, p<0.0001) and fentanyl (10.2% less LDH release, p<0.05) showed statistically significantly increased neuroprotection compared to no drug treated group. Compared to biphalin, LYS436 (p<0.05) and LYS739 (p<0.05) treated neurons released less LDH denoting a more potent effect than biphalin. LYS436 (p<0.0001) and LYS739 (p<0.0001) also showed better neuroprotection compared to fentanyl. In both assay, non-selective OR antagonist, NTX reversed the effect of most analogs and NTX did not show any significant effect compared to non-treated group.

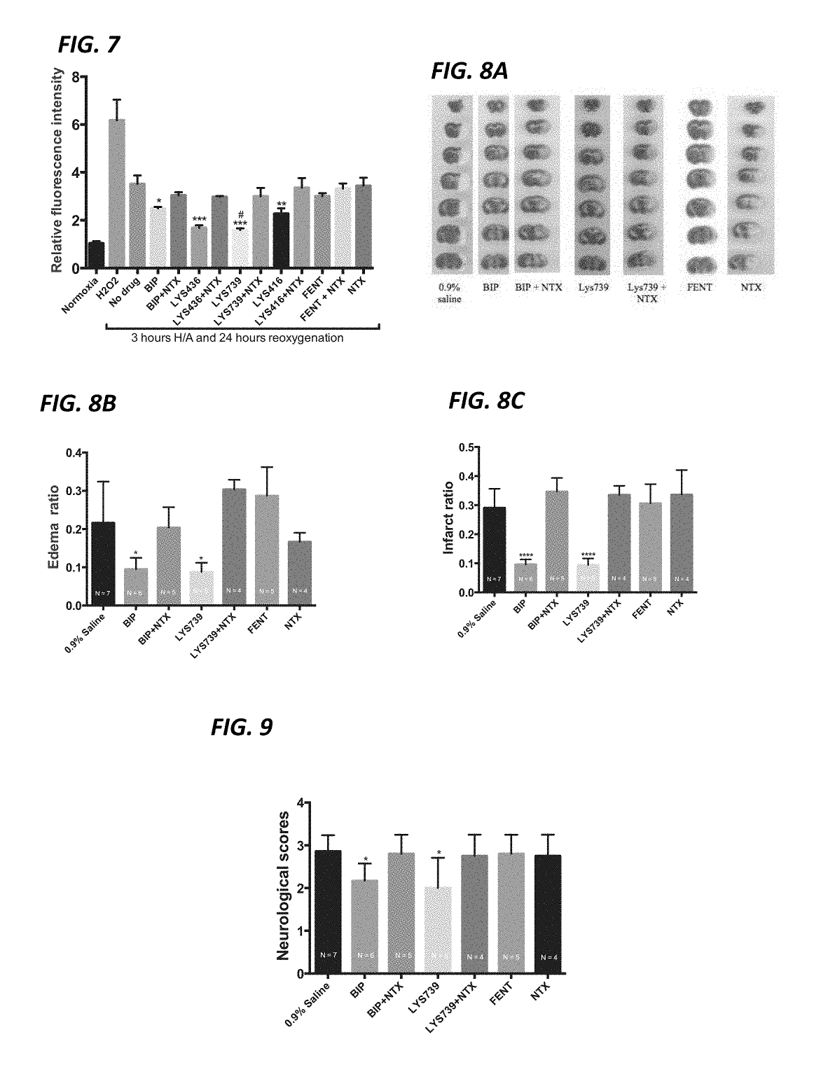

Generation of total ROS in primary cortical neuron exposed to 3 hr H/A and 24 hr reoxygenation in presence or absence of OR agonist fentanyl analogs and biphalin (10 nM) was assessed in this experiment (see FIG. 7). Total ROS generation was statistically significantly reduced when neuron were treated with LYS436 (52.2% less ROS production, p<0.001), LYS739 (54.4% less ROS production, p<0.001), LYS416 (35.0% less ROS production, p<0.01) and biphalin (29.1% less ROS production, p<0.05) compared to no drug treated group. The effect of LYS739 was significantly better (p<0.05) than that of biphalin. Both LYS436 (p<0.001) and LYS739 (p<0.001) significantly decreased ROS production compared to fentanyl. Non-selective OR antagonist NTX did not show significant decrease in ROS production compared to no drug treated group but it reversed the effect of OR agonists (except for fentanyl) used in this experiments.

As shown in FIG. 8A-C, The effect of fentanyl analog LYS739, biphalin and fentanyl on brain edema formation (see FIG. 8B) and infarct volume (FIG. 8C) after focal brain ischemia induced by 1 hr occlusion followed by 24 hr reperfusion. Compared to the vehicle treated group LYS739 produced a 59.45% reduction in edema formation, p<0.05 and biphalin produced a 56.17% reduction in edema formation, p<0.05 that was statistically significantly when administered 10 min after reperfusion at a dose of 5 mg/kg in saline (i.p.). Fentanyl (0.2 mg/kg, 10 minute post reperfusion) and/or antagonist NTX (1 mg/kg, 10 min before stroke) did not show any significant reduction in edema formation compared to saline treated group. LYS739 produced a 67.7-% reduction in infarct ratio, p<0.0001) and biphalin produced a 67.0% reduction in infarct ratio, p<0.0001 that were statistically significant compared to saline treated group. Again, fentanyl and NTX did not show any improvement in terms of infarct ratio. For both edema formation and infarction volume, NTX reversed the effect of both LYS739 and biphalin. Mean cerebral blood flow reduction±SEM in ischemic brain for saline group 80.7±1.2%, BIP 81.1±1.3%, BIP+NTX 79.7±2.0%, LYS739 82.1±1.2%, LYS739+NTX 80.6±2.2%, FENT 80.4±1.6%, NTX 76.9±2.0%.

Twenty-four hours after the reperfusion neurological score was evaluated in the experimental groups (see FIG. 9). LYS739 (30.4% improvement, p<0.05) and biphalin (25.5% improvement, p<0.05) significantly improved the neurological score compared to saline treated control group. Fentanyl or OR antagonist NTX did not improve any neurological score under same experimental conditions. NTX reversed the effect of both LYS739 and biphalin but the effects were not statistically significant.

Locomotor activity (horizontal activity, vertical activity, total distance, rest time, stereotype counts and number of movements) was evaluated 24 hr after reperfusion in experimental animals (Table 8). Before the start of surgery all animals went through locomotor evaluation to get the baseline.□ Both LYS739 and biphalin (5 mg/kg, 10 min post reperfusion, i.p.) statistically significantly improved all the locomotor parameters compared to saline treated control animals. When compared the effect of LYS739 to that of biphalin most of the parameter were improved although they were not statistically significant except for vertical activity (p<0.05). But, in comparison to fentanyl treated group, both LYS739 and biphalin showed better locomotor activity and the effects were statistically significant. Non-selective OR antagonist NTX did not improve any locomotor parameters.

Table 8 shows measurement of locomotor activity 24 h after stroke and drug treatments. Data represent the mean±S.E.M. of 4-5 independent determinations; numbers indicated in parenthesis in the line of the table columns donate to the number of experimental animals per group. ‘*’ Compared to Saline treated group—*p<0.05; **p<0.01; ***p<0.001; ****p<0.0001; ‘#’ Compared to biphalin treated group—#p<0.05; ##p<0.01; ###p<0.001; ####p<0.0001; ‘ϕ’ Compared to fentanyl treated group—ϕp<0.05; ϕϕp<0.01; ϕϕϕp<0.001; ϕϕϕϕp<0.0001

TABLE 8

| | | 0.9% | | BIP + | | LYS739 + | | |
|---|---|---|---|---|---|---|---|---|
| | Sham | Saline | BIP | NTX | LYS739 | NTX | FENT | NTX |
| Parameters | (N = 7) | (N = 7) | (N = 7) | (N = 5) | (N = 4) | (N = 4) | (N = 4) | (N = 4) |
| Horizontal | 1300 ± 230 | 150 ± 34 | 760 ± 75 | 390 ± 130 | 1000 ± 160 | 120 ± 9 | 140 ± 50 | 160 ± 36 |
| Activity | **** | ## | ** | | *** | ## | # | # |
| | # | | Φ | | ΦΦΦ | | | |
| Vertical | 36 ± 8 | 0 | 28 ± 3 | 20 ± 2 | 50 ± 13 | 0 | 3 ± 3 | 1.8 ± 1 |
| Activity | ** | ## | ** | | **** | ## | # | ## |
| | | Φ | | | # | | | |
| | | | | | ΦΦΦΦ | | | |
| Total | 1100 ± 280 | 17 ± 5 | 650 ± 81 | 88 ± 64 | 680 ± 110 | 8 ± 2 | 15 ± 6 | 45 ± 16 |
| Distance | **** | # | ** | # | ** | ## | ## | # |
| (CM) | | | ΦΦ | | ΦΦ | | | |
| No. of | 75 ± 16 | 10 ± 2 | 42 ± 6 | 19 ± 6 | 6 ± 17 | 6 ± 2 | 6 ± 1 | 11 ± 3 |
| Movements | ▪▪▪▪▪▪ | # | *□ | | ** | # | # | |
| | # | | Φ | | ΦΦ | | | |
| Stereotypy | 970 ± 110 | 62 ± 12 | 390 ± 46 | 230 ± 71 | 340 ± 76 | 39 ± 2 | 83 ± 35 | 77 ± 13 |
| Counts | **** | ## | ** | | * | ## | # | # |
| | #### | | Φ | | | | | |
| Rest Time | 220 ± 19 | 300 ± 1 | 240 ± 7 | 270 ± 7 | 220 ± 14 | 300 ± 27 | 300 ± 1 | 300 ± 2 |
| (Seconds) | **** | ## | ** | | **** | ### | ### | ## |
| | | | ΦΦΦ | | ΦΦΦΦ | | | |

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A multifunctional opioid receptor ligand (ORL) according to Formula 1: Aaa-Bbb-Ccc-Ddd(X)-Eee, wherein
Aaa is selected from 2′-6′-dimethyltyrosine (Dmt), Tyrosine (Tyr), Tmt, Phe, Dmp, and Mdp;
Bbb is selected from D-Alanine (D-Ala), Alanine (Ala), D-Norleucine (D-Nle), Norleucine (Nle), Proline (Pro), D-Proline (D-Pro), Arginine (Arg), D-Arginine (D-Arg), and tetrahydroisoquinoline-3-carboxylic acid (Tic), and D-Tic;
Ccc is selected from Gly, Phenylalanine(X) (Phe(X)), Trp, and naphthylalanine (Nal) or is absent;
Ddd(X) is Gly, Phe(X), Trp, Nal, or Lys; and
Eee comprises N-phenyl-N-piperidin-4-ylpropionamide-R (Ppp(R)) wherein X and R both comprise a halogen, X is selected from H, F, Cl, and Br, R is selected from F, Cl, and Br;
wherein the multifunctional ORL has agonist activity at mu opioid receptor (MOR), agonist activity at delta opioid receptor (DOR), and antagonist activity at kappa opioid receptor (KOR).

2. The ORL of claim 1, wherein R is selected from 3-Cl, 4-Cl, 3-F, 4-F, and 2,4-diCl.

3. A multifunctional opioid receptor ligand (ORL) according to Formula 4: Aaa-Bbb-Ccc-Ddd(X)-Yyy(n)-Eee, wherein
Aaa is selected from 2′-6′-dimethyltyrosine (Dmt), Tyrosine (Tyr), Tmt, Phe, Dmp, and Mdp;
Bbb is selected from D-Alanine (D-Ala), D-Norleucine (D-Nle), Proline (Pro), and D-Arginine (D-Arg), tetrahydroisoquinoline-3-carboxylic acid (Tic), D-Tic;
Ccc is selected from Gly, Phenylalanine(X) (Phe(X)), Trp, and naphthylalanine (Nal) or is absent, wherein X is a halogen;
Ddd(X) is Gly, Phe(X), Trp, Nal, or Lys, wherein X is a halogen;
Yyy is selected from one or a combination of Leu, Met, Lys, Arg, or lie, and
Eee is a 4-anilidopiperidine moiety;
wherein n=1, 2, 3, 4, 5, 6, 7, or 8;
wherein X is selected from H, F, Cl, and Br;
wherein the multifunctional ORL has agonist activity at mu opioid receptor (MOR), agonist activity at delta opioid receptor (DOR), and antagonist activity at kappa opioid receptor (KOR).

4. The ORL of claim 3, wherein the 4-anilidopiperidine moiety comprises Ppp.

5. The ORL of claim 4, wherein Ppp comprises Ppp(R), wherein R comprises a halogen.

6. The ORL of claim 5, wherein R is selected from 3-Cl, 4-Cl, 3-F, 4-F, and 2,4-diCl.

7. The ORL of claim 3, wherein the ORL is SEQ ID NO: 39, SEQ ID NO: 40, or SEQ ID NO; 41.

8. A multifunctional opioid receptor ligand (ORL) according to Formula 6: Aaa-Pro-Ccc-Phe(X)-Eee, wherein
Aaa is selected from Tyr or 2′-6′-dimethyltyrosine (Dmt);
Ccc is selected from Trp, Phe, Gly, or Phe(X);
Eee is a 4-anilidopiperidine moiety, and
X is selected from F, Cl, or Br.

9. The ORL of claim 8, wherein the 4-anilidopiperidine moiety comprises Ppp.

10. The ORL of claim 9, wherein Ppp comprises Ppp(R), wherein R comprises a halogen.

11. The ORL of claim 10, wherein R is selected from 3-Cl, 4-Cl, 3-F, 4-F, and 2,4-diCl.

12. The ORL of claim 8, wherein the ORL is selected from SEQ ID NO: 27, SEQ ID NO: 28, SEQ ID NO: 29, or SEQ ID NO: 30.


SEQUENCE LISTING (44 sequences)

SEQ ID NO: 1 - protein, 5 aa, Homo sapiens
YGGFL

SEQ ID NO: 2 - protein, 5 aa, Homo sapiens
YGGFM

SEQ ID NO: 3 - protein, 17 aa, Homo sapiens
YGGFLRRIRPKLKWDNQ

SEQ ID NO: 4 - protein, 13 aa, Homo sapiens
YGGFLRRIRPKLK

SEQ ID NO: 5 - protein, 8 aa, Homo sapiens
YGGFLRRI

SEQ ID NO: 6 - protein, 13 aa, Homo sapiens
YGGFLRRNFLVVT

SEQ ID NO: 7 - protein, 4 aa, Artificial Sequence
  Peptide has C-terminal Amidation
  SITE (2)..(2): Xaa refers to D-Alanine
  MOD_RES (4)..(4): F is amidated with -NH2, e.g., F is Phe-NH2
YAGF

SEQ ID NO: 8 - protein, 4 aa, Artificial Sequence
  Peptide has C-terminal amidation
  MISC_FEATURE (2)..(2): Ala is D-Alanine
  MOD_RES (4)..(4): F is amidated with N-phenyl-N-piperidin-4-ylpropionamide
YAGF

SEQ ID NO: 9 - protein, 4 aa, Artificial Sequence
  peptide has C-terminal amidation
  MISC_FEATURE (1)..(1): Xaa is 2,6-dimethyltyrosine
  MISC_FEATURE (2)..(2): Ala is D-Alanine
  MOD_RES (4)..(4): Phe is amidated with N-phenyl-N-piperidin-4-ylpropionamide
XAGF

SEQ ID NO: 10 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Norleucine
  MOD_RES (4)..(4): Phe is amidated with N-phenyl-N-piperidin-4-ylpropionamide
  MOD_RES (4)..(4): Phe halogenated with Fluorine
XXGF

SEQ ID NO: 11 - protein, 4 aa, Artificial Sequence
  Peptide has C-terminal amidation
  MISC_FEATURE (2)..(2): Xaa is D-Norleucine
  MOD_RES (4)..(4): Phe is amidated with -NH2
  MOD_RES (4)..(4): Phe is halogenated with fluorine
YXGF

SEQ ID NO: 12 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is N-Norleucine
  MOD_RES (4)..(4): Phe is amidated with -NH2
  MOD_RES (4)..(4): Phe is halogenated with Fluorine
XXGF

SEQ ID NO: 13 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Norleucine
  MOD_RES (4)..(4): Phe is amidated with -NH2
XXGF

SEQ ID NO: 14 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Norleucine
  MOD_RES (4)..(4): Phe is amidated with N-phenyl-N-piperidin-4-ylpropionamide
XXGF

SEQ ID NO: 15 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Norleucine
  MOD_RES (4)..(4): Phe is halogenated with Cl
  MOD_RES (4)..(4): Phe is amidated with N-phenyl-N-piperidin-4-ylpropionamide
XXGF

SEQ ID NO: 16 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Tic (D-tetrahydroisoquinoline-3-carboxylic acid)
  MISC_FEATURE (3)..(3): Xaa is absent
  MOD_RES (4)..(4): Phe is amidated with N-phenyl-N-piperidin-4-ylpropionamide
XXXF

SEQ ID NO: 17 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Norleucine
  MOD_RES (4)..(4): Phe is halogenated with Cl
  MOD_RES (4)..(4): Phe is amidated with -NH2
XXGF

SEQ ID NO: 18 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Norleucine
  MOD_RES (4)..(4): Phe is halogenated with Br
  MOD_RES (4)..(4): Phe is amidated with N-phenyl-N-piperidin-4-ylpropionamide
XXGF

SEQ ID NO: 19 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Norleucine
  MISC_FEATURE (3)..(3): Xaa is homocysteine
  MOD_RES (4)..(4): Phe is halogenated with fluorine
  MOD_RES (4)..(4): Phe is amidated with N-phenyl-N-piperidin-4-ylpropionamide
XXXF

SEQ ID NO: 20 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is homocysteine
  MOD_RES (4)..(4): Phe is halogenated with fluorine
  MOD_RES (4)..(4): Phe is amidated with N-phenyl-N-piperidin-4-ylpropionamide
XXGF

SEQ ID NO: 21 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Arg is D-Arginine
  MOD_RES (4)..(4): Gly is amidated with N-phenyl-N-piperidin-4-ylpropionamide
XRFG

SEQ ID NO: 22 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Arg is D-Arginine
  MISC_FEATURE (3)..(3): Xaa is 1-naphthylalanine
  MOD_RES (4)..(4): Gly is amdiated with N-phenyl-N-piperidin-4-ylpropionamide
XRXG

SEQ ID NO: 23 - protein, 4 aa, Artificial Sequence
  Peptide has a C-terminal amidation
  MISC_FEATURE (1)..(1): Xaa is 2,6-dimethyltyrosine
  MISC_FEATURE (2)..(2): Arg is D-Arginine
  MOD_RES (4)..(4): Lys is amidated with N-phenyl-N-piperidin-4-ylpropionamide
XRFK

SEQ ID NO: 24 - protein, 4 aa, Artificial Sequence
  Peptide has a C-terminal amidation
  MISC_FEATURE (1)..(1): Xaa is 2,6-dimethyltyrosine
  MISC_FEATURE (2)..(2): Xaa is D-Arginine
  MISC_FEATURE (2)..(2): Arg is D-Arginine
  MISC_FEATURE (3)..(3): Xaa is 1-naphthylalanine
  MOD_RES (4)..(4): Lys is amidated with N-phenyl-N-piperidin-4-ylpropionamide
XRXK

SEQ ID NO: 25 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Arginine
  MISC_FEATURE (2)..(2): Arg is D-Arginine
  MISC_FEATURE (3)..(3): Xaa is absent
  MISC_FEATURE (4)..(4): Xaa is 1-naphthylalanine
  MOD_RES (4)..(4): 1-naphthylalanine is amidated with N-phenyl-N-piperidin-4-ylpropionamide
XRXX

SEQ ID NO: 26 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Arginine
  MISC_FEATURE (2)..(2): Arg is D-Arginine
  MISC_FEATURE (3)..(3): Xaa is absent
  MOD_RES (4)..(4): Phe is halogenated with Cl
  MOD_RES (4)..(4): Phe is amidated with N-phenyl-N-piperidin-4-ylpropionamide
XRXF

SEQ ID NO: 27 - protein, 4 aa, Artificial Sequence
  Peptide has C-terminal amidation
  MISC_FEATURE (1)..(1): Xaa is 2,6-dimethyltyrosine
  MOD_RES (4)..(4): Phe is halogenated with Cl
  MOD_RES (4)..(4): Phe is amidated with N-phenyl-N-piperidin-4-ylpropionamide
XPWF

SEQ ID NO: 28 - protein, 4 aa, Artificial Sequence
  Peptide has C_terminal amidation
  MISC_FEATURE (1)..(1): Xaa is 2,6-dimethyltyrosine
  MOD_RES (4)..(4): Phe is halogenated with Cl
  MOD_RES (4)..(4): Phe is amidated with N-phenyl-N-piperidin-4-ylpropionamide
XPFF

SEQ ID NO: 29 - protein, 4 aa, Artificial Sequence
  Peptide has C-terminal amidation
  MISC_FEATURE (1)..(1): Xaa is 2,6-dimethyltyrosine
  MOD_RES (3)..(3): Phe is halogenated with Cl
  MOD_RES (4)..(4): Phe is halogenated with Cl
  MOD_RES (4)..(4): Phe is amidated with N-phenyl-N-piperidin-4-ylpropionamide
XPFF

SEQ ID NO: 30 - protein, 4 aa, Artificial Sequence
  Peptide has C-terminal amidation
  MISC_FEATURE (1)..(1): Xaa is 2,6-dimethyltyrosine
  MOD_RES (4)..(4): Phe is halogenated with Cl
  MOD_RES (4)..(4): Phe is amidated with N-phenyl-N-piperidin-4-ylpropionamide
XPGF

SEQ ID NO: 31 - protein, 4 aa, Artificial Sequence
  Peptide has C-terminal amidation
  MISC_FEATURE (2)..(2): Xaa is D-Alanine
  MISC_FEATURE (2)..(2): Ala is D-Alanine
  MOD_RES (4)..(4): Phe is amidated with a 4-anilidopiperidine moiety
YAGF

SEQ ID NO: 32 - protein, 4 aa, Artificial Sequence
  Peptide has a C-terminal amidation
  MISC_FEATURE (2)..(2): Xaa is D-Alanine
  MISC_FEATURE (2)..(2): Ala is D-Alanine
  MOD_RES (4)..(4): Phe is amidated with a 4-anilidopiperidine moiety
YAGF

SEQ ID NO: 33 - protein, 4 aa, Artificial Sequence
  Peptide has C-terminal amidation
  MISC_FEATURE (2)..(2): Xaa is D-Alanine
  MISC_FEATURE (2)..(2): Ala is D-Alanine
  MOD_RES (4)..(4): Phe is amidated with a 4-anilidopiperidine moiety
YAGF

SEQ ID NO: 34 - protein, 4 aa, Artificial Sequence
  Peptide has C-terminal amidation
  MISC_FEATURE (2)..(2): Xaa is D-Alanine
  MISC_FEATURE (2)..(2): Ala is D-Alanine
  MOD_RES (4)..(4): Phe is amidated with a 4-anilidopiperidine moiety
YAGF

SEQ ID NO: 35 - protein, 4 aa, Homo sapiens
  MOD_RES (4)..(4): Phe is amidated with -NH2
YPWF

SEQ ID NO: 36 - protein, 4 aa, Artificial Sequence
  Dermorphin analog
  MISC_FEATURE (2)..(2): Xaa is D-Arginine
  MISC_FEATURE (2)..(2): Arg is D-Arginine
  MOD_RES (4)..(4): Lys is amidated with -NH2
YRFK

SEQ ID NO: 37 - protein, 4 aa, Artificial Sequence
  Peptide has C-terminal amidation
  MOD_RES (4)..(4): PHE is amidated with N-phenyl-N-piperidin-4-ylpropionamide
YGGF

SEQ ID NO: 38 - protein, 5 aa, Artificial Sequence
  Peptide has C-terminal amidation
  MISC_FEATURE (1)..(1): Xaa is 2,6-dimethyltyrosine
  MISC_FEATURE (2)..(2): D-Norleucine (D-Nle)
  MOD_RES (4)..(4): Phe is halogenated with F
  MOD_RES (4)..(4): Phe is amidated with N-phenyl-N-piperidin-4-ylpropionamide
XXGFL

SEQ ID NO: 39 - protein, 4 aa, Artificial Sequence
  Peptide has C-terminal amidation
  MISC_FEATURE (1)..(1): 2,6-dimethyltyrosine
  MISC_FEATURE (2)..(2): tetrahydroisoquinoline-3-carboxylic acid (Tic)
  MOD_RES (4)..(4): Phe is amidated with N-phenyl-N-piperidin-4-ylpropionamide
XXGF

SEQ ID NO: 40 - protein, 4 aa, Artificial Sequence
  Peptide has C-terminal amidation
  MISC_FEATURE (1)..(1): 2,6-dimethyltyrosine
  MISC_FEATURE (2)..(2): tetrahydroisoquinoline-3-carboxylic acid
  MOD_RES (4)..(4): Phe is halogenated with F
  MOD_RES (4)..(4): Phe is amidated with N-phenyl-N-piperidin-4-ylpropionamide
XXGF

SEQ ID NO: 41 - protein, 4 aa, Artificial Sequence
  Peptide has C-terminal amidation
  MISC_FEATURE (1)..(1): 2,6-dimethyltyrosine
  MISC_FEATURE (2)..(2): tetrahydroisoquinoline-3-carboxylic acid
  MOD_RES (4)..(4): F is halogenated with Cl
  MOD_RES (4)..(4): F is amidated with N-phenyl-N-piperidin-4-ylpropionamide
XXGF

SEQ ID NO: 42 - protein, 4 aa, Artificial Sequence
  Peptide has C-terminal amidation
  MISC_FEATURE (1)..(1): 2,6-dimethyltyrosine
  MISC_FEATURE (2)..(2): D-Arginine
  MOD_RES (3)..(3): Phe is halogenated with Cl
  MOD_RES (4)..(4): L is amidated with N-phenyl-N-piperidin-4-ylpropionamide
XXFL

SEQ ID NO: 43 - protein, 4 aa, Artificial Sequence
  Peptide has C-terminal amidation
  MISC_FEATURE (1)..(1): 2,6-dimethyltyrosine
  MISC_FEATURE (2)..(2): D-Arginine
  MISC_FEATURE (3)..(3): Phe is halogenated with F
  MISC_FEATURE (4)..(4): L is amidated with N-phenyl-N-piperidin-4-ylpropionamide
XXFL

SEQ ID NO: 44 - protein, 4 aa, Artificial Sequence
  Peptide has C-terminal amidation
  MISC_FEATURE (1)..(1): D-Phenylalanine
  MISC_FEATURE (2)..(2): D-Phenylalanine
  MISC_FEATURE (3)..(3): D-Norleucine
  MISC_FEATURE (4)..(4): D-Lysine
  MOD_RES (4)..(4): D-Lys is amidated with N-phenyl-N-piperidin-4-ylpropionamide
XXXX